(19) United States
(12) Patent Application Publication
(10) Pub. No.: US 2020/0334597 A1
(43) Pub. Date: Oct. 22, 2020

(54) SYSTEMS AND METHODS FOR WORKFLOW PROCESSING

(71) Applicant: James Gelsin Marx, Las Vegas, NV (US)

(72) Inventor: James Gelsin Marx, Las Vegas, NV (US)

(21) Appl. No.: 16/694,835

(22) Filed: Nov. 25, 2019

Related U.S. Application Data
(63) Continuation of application No. 14/231,348, filed on Mar. 31, 2014.

(51) Int. Cl. G06Q 10/06; G06Q 50/22; G16H 40/20; G06F 40/284

(52) U.S. Cl. CPC G06Q 10/0633; G06F 40/284; G16H 40/20; G06Q 50/22

(57) ABSTRACT

Systems and methods for processing a workflow are disclosed. Certain embodiments relate to determining the richness of a requester's requisition data. Certain embodiments relate to determining the richness of an interpreter's interpretation data that is related to the requisition data. The richness of the requisition data and the interpretation data can be compared to determine whether the interpretation data is deficient. If the interpretation data is deficient, the interpreter can be notified so as to increase the value of the interpretation data to the requester. The richness of the requisition data can also be used to determine whether the subject data set would benefit from group collaboration, rather than interpretation by a single interpreter. The disclosed systems and methods have applications including, but not limited to, training, performance analysis, process improvement, and data analysis and data mining in workflows.

### PRIORITY AND INCORPORATION

Any and all applications for which a foreign or domestic priority claim is identified in the Application Data Sheet as filed with the present application are hereby incorporated by reference under 37 CFR 1.57. In particular, the present application is a continuation of U.S. application Ser. No. 14/231,348, filed Mar. 31, 2014, titled SYSTEMS AND METHODS FOR WORKFLOW PROCESSING.

### BACKGROUND

### Field

This disclosure relates generally to workflows and, more particularly, to training or quality improvement systems for workflows.

### Description of the Related Art

Many kinds of industries, businesses, and applications use software systems that manage the flow, progress, rate, or amount of work or data (the “workflow”) to and from an office, department, employee, individual, or group of individuals. For example, healthcare providers such as hospitals use workflow systems like Clinical Information Systems (CIS) and/or Hospital Information Systems (HIS) to manage administrative, financial, medical, and/or laboratory data; Radiology Information Systems (RIS) to store, manipulate, and distribute patient radiological data and imagery; and Picture Archiving and Communication Systems (PACS) for managing medical imaging aspects of the workflow.

Workflows can be used to increase productivity. For example, a primary provider can recommend that a sick patient have a medical image captured in order to facilitate diagnosis. An MRI acquires the image, and the image is sent to a PACS server. A clinician who specializes in making diagnoses from MRI can access the sick patient's image stored on the PACS system through a diagnostic review workstation. The clinician evaluates the image and makes an interpretation based on the image, entering the normal or abnormal interpretation into the workstation's interface. The image's interpretation is transmitted to a CIS. The primary provider can later query the CIS and view the diagnostic clinician's interpretation. The primary provider presents this interpretation to the patient. Because the acts of judging the image and making the interpretation are moved from the primary provider to the diagnostic clinician, the primary provider has more time to visit with other patients. Furthermore, the diagnostic clinician can efficiently review large numbers of images in series without having to interact with patients or their providers. PACS thus increases both the primary provider's and the diagnostic clinician's productivity.

Although workflows such as CIS, HIS, RIS, PACS, and others can increase productivity and can efficiently store and present data, these workflows have significant drawbacks. Serial workflows, like the diagnostic clinician's workflow described above, typically present a preponderance of normal data and only a very small percentage of abnormal data. This gives rise to a tendency to under-identify abnormal data in a workflow, particularly if the workflow is presented at a rapid pace. For example, a diagnostic clinician can use PACS to review a long series of radiological images to diagnose health conditions. Most of the images presented to the diagnostic clinician show normal results. Only a small number of images typically contain abnormalities. After reviewing long series of normal data, however, the diagnostic clinician can become bored, complacent, or fatigued and may tend to under-diagnose, over-diagnose, or misdiagnose subtle and perhaps even startling abnormalities presented in the workflow.

### SUMMARY

Systems and methods for processing a workflow are disclosed. The systems and method have application in a variety of workflow environments including, among other things and without limitation, medical diagnostics, such as radiology, interpreting satellite imagery, photo reconnaissance, industrial inspections, semiconductor processing, luggage inspections, and other applications discussed in this disclosure. Related workflow processing systems and methods are further described in U.S. Patent Application No. 61/016,892, filed Dec. 27, 2007, U.S. patent application Ser. No. 12/348,408, filed Dec. 23, 2008, U.S. Pat. No. 7,930,193, issued Apr. 19, 2011, and U.S. Pat. No. 7,937,277, issued May 3, 2011, each of which is hereby incorporated by reference in its entirety.

In at least one embodiment, a computer-implemented method for processing data is provided, the method comprising, with a computer system comprising one or more computing devices, receiving an electronic requisition comprising an order for an interpreter to interpret one or more files; parsing the electronic requisition to identify requisition words in the requisition; detecting which of the requisition words or variants thereof is/are present in a first database of keywords; respectively ranking or scoring the requisition word(s) or variant(s) thereof that is/are present in the first database; generating a cumulative requisition rank or score based on the rank(s) or score(s) of the requisition word(s) or variant(s); receiving an electronic interpretation generated by the interpreter, the electronic interpretation comprising interpretive data based on the one or more files; parsing the electronic interpretation to identify interpretation words in the interpretation; detecting which of the interpretation words or variants thereof is/are present in a second database of keywords; respectively ranking or scoring the interpretation word(s) or variant(s) thereof that is/are present in the second database; generating a cumulative interpretation rank or score based on the rank(s) or score(s) of the interpretation word(s) or variant(s); and determining whether the cumulative interpretation rank or score meets a threshold of the cumulative requisition rank or score. The foregoing method can also be implemented or embodied in a non-transitory, computer-readable medium and/or a system for processing data.

In various embodiments, the foregoing method has one, some or all of the following properties, as well as properties described elsewhere in this disclosure. The first database and the second database can be the same. The method can further comprise notifying the interpreter to modify or supplement the electronic interpretation when the cumulative interpretation rank or score does not meet the threshold. The method can further comprise detecting which of the interpretation words or variants thereof are contextual terms and adjusting the rank or score of any interpretation word that is adjacent or within a number of words of a contextual term in the electronic interpretation or the variant thereof. The method can further comprise determining a context of the electronic requisition and determining the rank or score of one or more interpretation words or variants thereof based on the context. The first database and the second database can be computer-processable collections of medical terms. Respectively ranking or scoring the requisition word(s) or variant(s) thereof can occur substantially simultaneous with detecting which of the requisition words or variants thereof is/are present in the first database of keywords. Respectively ranking or scoring the interpretation word(s) or variant(s) thereof can occur substantially simultaneous with detecting which of the interpretation words or variants thereof is/are present in the second database of keywords. Respectively ranking or scoring the interpretation word(s) or variant(s) thereof can comprise assigning an increasingly higher rank or score depending on a seriousness, abnormality, or criticality of a condition, finding, or variation indicated by the respective word or variant thereof.

In at least one embodiment, a system for processing files is also provided. The system can comprise some or all of the following components, as well as components described elsewhere in this disclosure: an unscreened files module, a prescreened files data module, a distribution module, at least one workstation, a comparison module, a feedback module, a cloaking module, a diagnostic output module, a feedback module, an alerting module, and a system monitoring module. For example, in some embodiments, the system comprises the unscreened files data structure, the prescreened files data structure, the distribution module, at least one workstation, the comparison module, and the feedback module. In at least one embodiment, the system comprises the unscreened files data structure, the distribution module, at least one workstation, and the diagnostic output module.

In various embodiments, the foregoing system has one, some or all of the following properties, as well as properties described elsewhere in this disclosure. The unscreened data structure can be configured to access unscreened files created by at least one imaging modality. The prescreened data structure can be configured to access prescreened files. The prescreened data structure can be configured to access an associated interpretation for each of the prescreened files.

A distribution module can comprise some or all of the following components, as well as components described elsewhere in this disclosure: a selection processor, a workflow queue generation processor, a transmission processor, a distribution monitoring processor, and a distribution override processor. For example, the distribution module can comprise the selection processor and the transmission processor. In at least one embodiment, the distribution module comprises the selection processor, the workflow queue generation processor, and the transmission processor. In at least one embodiment, the distribution module additionally comprises the distribution monitoring processor and the distribution override processor.

The selection processor can be configured to select one or more files from the prescreened files data structure. The selection processor can be configured to select one or more files from the unscreened data structure. The selection processor can configured to select a plurality of files from the prescreened files data structure and the unscreened files data structure. The workflow queue generation processor can be configured to generate a workflow queue. The workflow queue can comprise the selection of files. The transmission processor can be configured to transmit the workflow queue. The transmission processor can be configured to transmit the selection of files. The distribution monitoring processor can be configured to monitor the receipt of the prescreened files.

The distribution override processor can be in communication with the distribution monitoring processor or the selection processor. In some embodiments, the distribution override processor can be in communication with the distribution monitoring processor and the selection processor. The distribution override processor can be configured to prevent the selection processor from selecting from the prescreened files module when the distribution monitoring processor indicates that the receipt of the prescreened files exceeds a threshold value signifying an emergency condition or when an aggregated rank or score (described below) exceeds a threshold value signifying an emergency condition. Other override conditions are also appropriate for use herein.

A workstation can comprise some or all of the following components, as well as components described elsewhere in this disclosure: a receiving processor, an interpreter input, and an interpreter display. In at least one embodiment, the workstation comprises all of the preceding components. The receiving processor can be configured to receive the workflow queue from the distribution module. The receiving processor can be configured to receive the selection of medical files from the distribution module. The interpreter display can be configured to display the files in the workflow queue or in the selection of files. The interpreter display can be configured to serially display the files. The interpreter input can be configured to receive an interpreter interpretation associated with the file displayed on the interpreter display. The workstation can be configured to receive electronic tag data through the interpreter input. Examples of electronic tag data are, for example, Boolean, binary, ASCII data, and the like. The workstation can be configured to associate the electronic tag data with the file displayed on the interpreter display.

The system can comprise a central repository. The central repository can be a source of prescreened data. In some embodiments, the prescreened files data structure is configured to access files in the central repository. The central repository can be configured to receive a file associated with above-described electronic tag data.

The comparison module can be configured to receive the interpreter's interpretation from the at least one workstation. The comparison module can be configured to determine whether the interpreter's interpretation is associated with a prescreened file. The comparison module can generate comparison data if the interpreter's interpretation is associated with a prescreened file. In some embodiments, the comparison data can be generated by comparing the interpreter's interpretation with the associated interpretation.

The feedback module can be configured to receive the comparison data from the comparison module. The feedback module can be configured to transmit the comparison data to the workstation interpreter display.

The cloaking module can be configured to alter origination data associated with prescreened files. For example, the origination data can be altered such that prescreened files appear to originate from the above-mentioned at least one modality.

The diagnostic output module can be configured to receive the interpreter's interpretation from the at least one workstation. The diagnostic output module can generate a rank or score based on a textual evaluation of the interpreter's interpretation. The diagnostic output module can associate the rank or score with the file displayed on the interpreter display.

The feedback module can be configured to receive textual comment data from a requester linked with a certain file, such as the file displayed on the interpreter display. A requester can be linked with a file, for example, using data embedded in a file's header or metadata. As another example, a requester can be associated with the file with a database table. The feedback module can be configured to display the textual comment data on the interpreter display.

The alerting module can be configured to receive the rank or score from the diagnostic output module. The alerting process can be configured to transmit an alert to a requester linked with the associated file if the rank or score exceeds a threshold indicating a serious condition.

The system monitoring module can be configured to generate an aggregated rank or score with ranks or scores received from the diagnostic output module. The aggregated rank or score can be used to trigger override conditions. The aggregated rank or score can be used to adjust the amount of prescreened files distributed to an interpreter. For example, the ratio of unscreened files to prescreened files in the workflow queue can depend on the aggregated rank or score.

In at least one embodiment, a method for processing a workflow is provided. Production workflow data produced by at least one modality, wherein the production workflow data has not been previously characterized, is inserted into a file queue. Prescreened data, wherein the prescreened data has been previously characterized and the prescreened data comprises previous characteristics, is also inserted into the file queue. The file queue is transmitted for an interpretation. The received interpretation of the data comprised in the file queue is evaluated. Based at least in part on the evaluation, the amount of prescreened data inserted into the file queue is adjusted.

The method can comprise some or all of the following actions, as well as actions described elsewhere in this disclosure. Before inserting prescreened data into the file queue, the identification information of the prescreened data can be altered so that the prescreened data appears to be produced by at least one modality. The alteration may be performed centrally or locally. At least some of the interpreted production workflow data can be reused as prescreened data. The interpretation of some of the production workflow data may be reviewed and characterized. Some of that data may be reused as prescreened data. The evaluation of the interpretation of the data comprised in the queue comprises comparing the interpretation of the prescreened data with previous characteristics. The amount of the prescreened data inserted into the file queue may be adjusted based at least in part on the results of the comparison. For example, the amount of prescreened data inserted into the file queue may be increased when the results of the comparison are not successful. In addition, the results of the comparison may be communicated to the interpreter.

The evaluation of the received interpretation of the data comprised in the queue comprises assigning a rank or score based on complexity of characteristics of the production workflow data, with the rank or score being at least in part proportional to the complexity. The adjustment of the amount of the prescreened data inserted into the file queue may be at least in part inversely proportional to the assigned rank or score of the previous work product output. The evaluation of the received interpretation of the data comprised in the queue may allow enhancing the use of data analysis and data mining capabilities of the workflow. Data analysis and data mining may be used to improve, forecast, and predict interpreters' accuracy and efficiency. At least some of the received interpretation of the data comprised in the file queue may be marked as “unknown,” when the interpreter is unsure of the interpretation. At least some of the data marked as “unknown” may be reinserted into the file queue, and transmitted for another attempt at the interpretation by the same or another interpreter.

In at least one embodiment, a computer-implemented method for processing data is provided. The method comprises, with a computer system comprising one or more computing devices, receiving, from a interpreter's workstation, interpretive data created by the interpreter during review of a set of one or more image files that have not been previously reviewed by the interpreter, wherein the interpretive data comprises a characterization of structures or findings that the interpreter observed in the set of one or more image files. The method also comprises parsing text in the interpretive data for keywords and applying a scoring or ranking algorithm to the interpretive data based on the keywords, such that a score or rank is assigned to the interpretive data, wherein the algorithm assigns a neutral or negative score or rank to normal findings or structures, a lower score or rank to less severely abnormal or critical findings or structural variants, and a higher score or rank to more severely abnormal or critical findings or structural variants, such that a higher score or rank is assigned to increasingly severely abnormal or critical findings or structural variants.

The method also comprises taking an action based on the score or rank. The action can include one or both of the following: automatically transmitting, to a requesting entity's computer, PDA, or another computer accessible by the requesting entity's workstation or another computing device accessible by the requesting entity, based on the score or rank, a first electronic communication concerning the abnormality or criticality of the interpretive data on an urgent basis; or generating an aggregate score or rank from assigned scores or ranks, and, based on the presence of a low aggregate score or rank, automatically notifying the interpreter to take a break or automatically sending prescreened data to the interpreter, wherein the prescreened data comprises one or more image files that have been previously reviewed by a reviewing entity other than the interpreter. As used herein, “automatically” means to perform a function such that, once the function is initiated, the function is performed by a machine, without the need for manually performing the function.

The foregoing method can have one, some, or all of the following properties, as well as properties described elsewhere in this disclosure. Transmitting can comprise transmitting the first electronic communication during the interpreter's review of the set of one or more image files. Transmitting can comprise transmitting the first electronic communication after the interpreter's review of the set of one or more image files. The method can further comprise receiving an electronic acknowledgment of the first electronic communication from the interpreter. The method can further comprise watching for the electronic acknowledgment; and when not received within a time period, transmitting a second electronic communication concerning the abnormality or criticality of the interpretive data. The second electronic communication can be transmitted using a different communication medium than the first electronic communication. Transmitting the first electronic communication can comprise transmitting when the rank exceeds a threshold indicating a serious condition. Applying the scoring or ranking algorithm can assign the rank by accounting for the context of the image file. The aggregate score or rank can be related to the arithmetic sum, the moving average, or some other aggregation of the interpreter's rank over time.

In at least one embodiment, a computer-implemented method for processing data is provided. With a computer system comprising one or more computing devices, an electronic requisition comprising an order for an interpreter to interpret one or more files is received; the electronic requisition is parsed to identify requisition words in the requisition; it is detected which of the requisition words or variants thereof is/are present in a first database of keywords; the requisition word(s) or variant(s) thereof that is/are present in the first database is/are respectively ranked or scored; a cumulative requisition rank or score based on the rank(s) or score(s) of the requisition word(s) or variant(s) is generated; an electronic interpretation generated by the interpreter is received, the electronic interpretation comprising interpretive data based on the one or more files; the electronic interpretation is parsed to identify interpretation words in the interpretation; it is detecting which of the interpretation words or variants thereof is/are present in a second database of keywords; the interpretation word(s) or variant(s) thereof that is/are present in the second database is/are respectively ranked or scored; a cumulative interpretation rank or score based on the rank(s) or score(s) of the interpretation word(s) or variant(s) is generated; and it is determining whether the cumulative interpretation rank or score meets a threshold of the cumulative requisition rank or score.

The foregoing method can comprise one, some, or all of the following actions, as well as actions described elsewhere in this disclosure. The first database and the second database can be the same. The method can further comprise notifying the interpreter to modify or supplement the electronic interpretation when the cumulative interpretation rank or score does not meet the threshold. The method can further comprise detecting which of the interpretation words or variants thereof are contextual terms and adjusting the rank or score of any interpretation word that is adjacent or within a number of words of a contextual term in the electronic interpretation or the variant thereof. The method can further comprise determining a context of the electronic requisition and determining the rank or score of one or more interpretation words or variants thereof based on the context. The first database and the second database can be computer-processable collections of medical terms. Respectively ranking or scoring the requisition word(s) or variant(s) thereof can occur substantially simultaneous with detecting which of the requisition words or variants thereof is/are present in the first database of keywords. Respectively ranking or scoring the interpretation word(s) or variant(s) thereof can occur substantially simultaneous with detecting which of the interpretation words or variants thereof is/are present in the second database of keywords. Respectively ranking or scoring the interpretation word(s) or variant(s) thereof can comprise assigning an increasingly higher rank or score depending on a seriousness, abnormality, or criticality of a condition, finding, or variation indicated by the respective word or variant thereof.

In at least one embodiment, a computer system including one or more computer devices is provided. The system comprises an requisition receiver configured to receive an electronic requisition comprising an order for an interpreter to interpret one or more files; a requisition parser configured to identify requisition words in the requisition; a requisition detector configured to detect which of the requisition words or variants thereof is/are present in a first database of keywords; a requisition ranking or scoring module configured to respectively rank or score the requisition word(s) or variant(s) thereof that is/are present in the first database; a cumulative requisition ranking or scoring module configured to determine a cumulative rank or score for the electronic requisition based on the rank(s) or score(s) of the requisition word(s) or variant(s); an interpretation receiver configured to an electronic interpretation generated by the interpreter, the electronic interpretation comprising interpretive data based on the one or more files; an interpretation parser configured to parse the electronic interpretation to identify interpretation words in the interpretation; an interpretation detector configured to detect which of the interpretation words or variants thereof is/are present in a second database of keywords; an interpretation ranking or scoring module configured to respectively rank or score the interpretation word(s) or variant(s) thereof that is/are present in the second database; a cumulative interpretation ranking or scoring module configured to generate a cumulative interpretation rank or score based on the rank(s) or score(s) of the interpretation word(s) or variant(s); and a determiner configured to determine whether the cumulative interpretation rank or score meets a threshold of the cumulative requisition rank or score.

The foregoing system can comprise one, some, or all of the following properties, as well as properties described elsewhere in this disclosure. The first database and the second database can be the same. The system can further comprise a notifier configured to notify the interpreter to modify or supplement the electronic interpretation when the cumulative interpretation rank or score does not meet the threshold. The system can further comprise a first context adjuster configured to detect which of the interpretation words or variants thereof are contextual terms and adjust the rank or score of any interpretation word that is adjacent or within a number of words of a contextual term in the electronic interpretation or the variant thereof. The system can further comprise a second context adjuster configured to determine a context of the electronic requisition and determine the rank or score of one or more interpretation words or variants thereof based on the context. The first database and the second database can be computer-processable collections of medical terms. The interpretation ranking or scoring module can be further configured to assign an increasingly higher rank or score depending on a seriousness, abnormality, or criticality of a condition, finding, or variation indicated by the respective word or variant thereof.

In at least one embodiment, a non-transitory computer-readable medium that stores executable instructions that direct an electronic host device to perform a method for processing data is provided. The method comprises receiving an electronic requisition comprising an order for an interpreter to interpret one or more files; parsing the electronic requisition to identify requisition words in the requisition; detecting which of the requisition words or variants thereof is/are present in a first database of keywords; respectively ranking or scoring the requisition word(s) or variant(s) thereof that is/are present in the first database; generating a cumulative requisition rank or score based on the rank(s) or score(s) of the requisition word(s) or variant(s); receiving an electronic interpretation generated by the interpreter, the electronic interpretation comprising interpretive data based on the one or more files; parsing the electronic interpretation to identify interpretation words in the interpretation; detecting which of the interpretation words or variants thereof is/are present in a second database of keywords; respectively ranking or scoring the interpretation word(s) or variant(s) thereof that is/are present in the second database; generating a cumulative interpretation rank or score based on the rank(s) or score(s) of the interpretation word(s) or variant(s); and determining whether the cumulative interpretation rank or score meets a threshold of the cumulative requisition rank or score.

The foregoing non-transitory computer-readable medium can comprise one, some, or all of the following properties, as well as properties described elsewhere in this disclosure. The method can further comprise detecting which of the interpretation words or variants thereof are contextual terms and adjusting the rank or score of any interpretation word that is adjacent or within a number of words of a contextual term in the electronic interpretation or the variant thereof. The method can further comprise determining a context of the electronic requisition and determining the rank or score of one or more interpretation words or variants thereof based on the context. Respectively ranking or scoring the interpretation word(s) or variant(s) thereof can comprise assigning an increasingly higher rank or score depending on a seriousness, abnormality, or criticality of a condition, finding, or variation indicated by the respective word or variant thereof.

For purposes of summarizing the embodiments and the advantages achieved over the prior art, certain items and advantages are described herein. Of course, it is to be understood that not necessarily all such items or advantages may be achieved in accordance with any particular embodiment. Thus, for example, those skilled in the art will recognize that the inventions may be embodied or carried out in a manner that achieves or optimizes one advantage or group of advantages as taught or suggested herein without necessarily achieving other advantages as may be taught or suggested herein. The flow charts described herein do not imply a fixed order to the steps, and embodiments of the invention may be practiced in any order that is practicable.

### BRIEF DESCRIPTION OF THE DRAWINGS

A general architecture that implements the various features of the disclosed systems and methods will now be described with reference to the drawings. The drawings and the associated descriptions are provided to illustrate embodiments and not to limit the scope of the disclosure. Throughout the drawings, reference numbers are re-used to indicate correspondence between referenced elements. In addition, the first digit of each reference number indicates the figure in which the element first appears.

FIG. 1 is a block diagram illustrating a workflow system.

FIG. 2 illustrates sources of unscreened data.

FIG. 3 illustrates insertion of prescreened data into a workflow.

FIG. 4 illustrates operation of a workflow distribution module.

FIG. 5 illustrates processing of a file queue comprising prescreened data.

FIG. 6A and FIG. 6B illustrate providing notification for the interpretation of prescreened data.

FIGS. 7 and 8 is a block diagram illustrating the integration of a central repository into a workflow.

FIG. 9A and FIG. 9B illustrate using a central repository for facilitating a consensus or collaborative diagnoses.

FIG. 10 illustrates provision of feedback to the interpreter.

FIG. 11 is a block diagram illustrating a workflow system.

FIG. 12 is another block diagram illustrating a workflow system.

These and other features will now be described with reference to the drawings summarized below. These drawings and the associated description are provided to illustrate preferred embodiments of the invention, and not limit the scope of the invention.

### DETAILED DESCRIPTION

This disclosure describes various embodiments of workflow processing systems and methods. The disclosed systems and methods have applications including, but not limited to, training, performance analysis, process improvement, and data analysis and data mining in workflows.

As explained above, it is desirable that an interpreter accurately process data presented in a workflow. However, workflows typically present a preponderance of normal data and only a very small percentage of abnormal data. After reviewing long series of normal data, an interpreter can become bored, complacent, or fatigued and may tend to under-interpret, over-interpret, or misinterpret subtle and perhaps even startling abnormalities presented in the workflow, particularly if the workflow is presented at a rapid pace.

Various embodiments include the realization that workflow processing can be improved by assessing the richness of an interpreter's interpretive output and adjusting the processing based on that assessment. Depending on the application, richness can reflect, among other things, the descriptiveness, amount of detail, seriousness, or criticality of the interpretive output, the difficulty in interpreting the underlying data, or the quantity or rarity of the condition(s) in the underlying data. A richer interpretive output may be more descriptive, have more details, or have more serious or critical findings than a less rich interpretive output. Data that is difficult to interpret may result in richer interpretive output than data than is easy to interpret. Data including a rare condition or a large number of conditions may result in richer interpretive output than data with common or only a few conditions. For example, when the interpretive output is part of a radiological report, a finding that “lung fields are clear” is less serious or critical (and, thus, less rich) than a finding of “infiltrate,” which is less serious or critical (and, thus, less rich) than a finding of “pleural effusion.” A finding of “subdiaphragmatic air,” a sign of a ruptured bowel or stomach, is still more serious or critical (and, thus, richer).

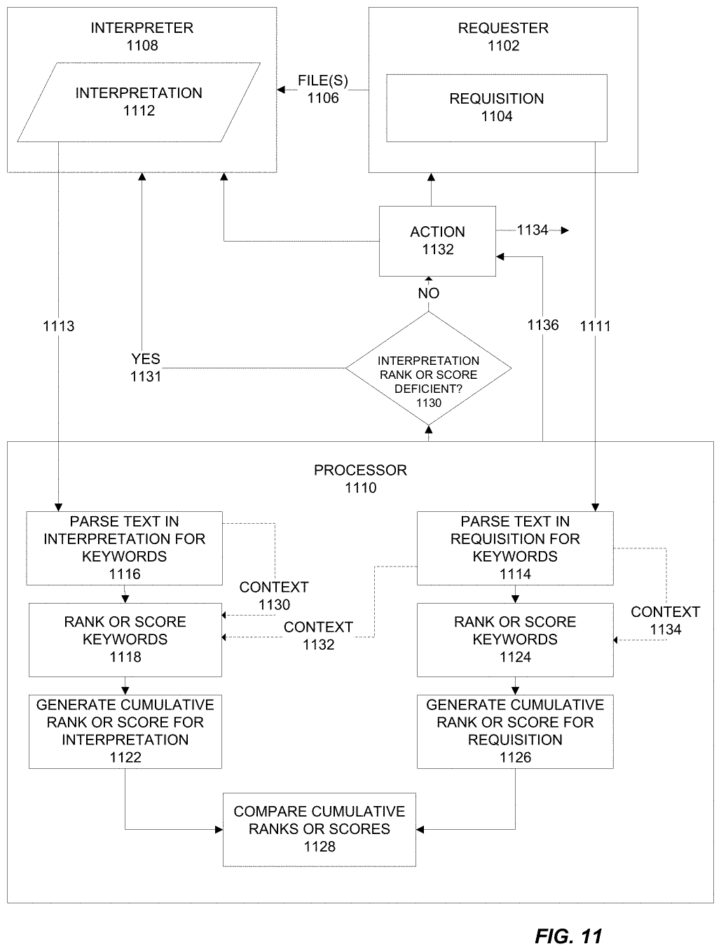

An example system and method for processing data is shown in FIG. 11. With a computer, a requester 1102 creates an electronic requisition 1104. A requisition 1104 is an order for an interpreter 1108 to interpret one or more files 1106. In at least one embodiment, the computer is a conventional computer that is equipped with a conventional modem. In other embodiments, the computer can be any suitable device that allows the interpreter to interact with the system, by way of example, a personal digital assistant, a smart phone, an electronic scanner, a computer workstation, a local area network of individual computers, an interactive wireless communications device, an interactive television, a transponder, or the like. Preferably, at least part of the requisition 1104 is textual and comprises parseable components, such as words. For example, with a networked personal computer, an ordering physician or the ordering physician's proxy can enter data in an electronic radiology requisition form and submit the form, thereby creating an electronic requisition 1104 for a radiologist to interpret image files in a radiological study. In some embodiments, at least part of the requisition 1104 is verbal and comprises words.

Based on the requisition 1104, the desired one or more files 1106 are given or sent to the interpreter 1108, or retrieved by the interpreter 1108 via a communications medium. In at least one embodiment, the communications medium is the Internet which is a global network of computers. In other embodiments, the communications medium can be any communication system including by way of example: dedicated communication lines, telephone networks, wireless data transmission systems, two-way cable systems, computer networks, and the like. For example, a PACS can push a radiological study comprising certain image files to the radiologist, or the radiologist can pull the desired radiological study from the PACS over dedicated communication lines.

Optionally, in certain embodiments, data from the requisition 1104 can be sent to a processor 1110 via a communications medium, as shown by line 1111. Suitable communications media are described above. The communications medium can be the same medium, or a different medium, used for transmitting files 1106 to the interpreter 1108. The processor 1110 can comprise one or more computers. The processor 1110 can comprise, by way of example, program logic or other substrate configurations representing data and instructions, which operate as described herein. In some embodiments, the processor 1110 can comprise controller circuitry, processor circuitry, general purpose single-chip or multi-chip microprocessors, digital signal processors, embedded microprocessors, microcontrollers, and/or the like.

With a computer, the interpreter 1108 generates an interpretation 1110 comprising interpretive data based on the file(s) 1106. In at least one embodiment, the computer is a conventional computer that is equipped with a conventional modem. In other embodiments, the computer can be any suitable device that allows the interpreter to interact with the system, by way of example, a personal digital assistant, a smart phone, an electronic scanner, a computer workstation, a local area network of individual computers, an interactive wireless communications device, an interactive television, a transponder, or the like. Preferably, at least part of the interpretation1110 is textual and comprises parseable components, such as words. For example, the radiologist at a computer workstation can generate a narrative radiological report based on the radiological study. In some embodiments, at least part of the interpretation 1110 is verbal and comprises words, such as an electronic dictation.

Certain data from the interpretation 1112 can be sent to the processor 1110 via a communications medium, as shown by line 1113. Suitable communications media are described above. The communications medium can be the same medium, or a different medium, used for transmitting files 1106 to the interpreter 1108 or sending data from the requisition 1104 to the processor 1110.

As shown in box 1116, the processor 1110 parses the interpretation 1112 with program logic executing one or more algorithms. The program logic can advantageously be implemented as one or more modules. The modules can advantageously be configured to execute on one or more processors. The modules can comprise, but are not limited to, any of the following: software or hardware components such as software object-oriented software components, class components and task components, processes methods, functions, attributes, procedures, subroutines, segments of program code, drivers, firmware, microcode, circuitry, data, databases, data structures, tables, arrays, or variables.

An example algorithm for parsing 1116 the interpretation 1112 comprises evaluating determining whether a word in the interpretation 1112 matches a keyword in a database. The parsing can also comprise determining whether a word in the interpretation 1112 matches a variant (such as a plural, gerund form, or even a common misspelling) of the keyword. In the foregoing disclosure, it should be understood the term “keywords” can include such variants. For example, the algorithm can evaluate words in text by regular expression processing. The algorithm can evaluate words in a verbal interpretation 1112 by voice recognition. In short, the keywords in the database indicate relevant, application-specific terms. The keywords in the database can be imported or otherwise adapted from application-specific nomenclature sources. For example, SNOMED CT is a computer-processable collection of medical terms used in clinical documentation and reporting. RadLex is a lexicon for radiology information resources. Keywords for radiological applications can be adapted from SNOMED CT and/or RadLex. Other appropriate sources, such as a list of site-specific vocabulary can also be used to populate the keywords in the database.

When requisition 1104 data has been sent to the processor 1110, as shown by line 1111, the processor 1110 can parse 1114 the requisition 1104 data with program logic executing one or more algorithms. An example algorithm for parsing 1114 comprises determining whether a word in requisition 1104 data text is stored in a database comprising keywords, similar to the algorithm described above. The keyword database associated with the requisition 1104 data can be the same as, or different from, the keyword database associated with the interpretation 1112. As discussed above, the algorithm can also involve identifying variants of the keywords, for instance by regular expression matching. In the following discussion, it should be understood that the term “keywords” includes such variants.

As shown in box 1118, the keywords in the interpretation 1112 (that is, the words in the interpretation 1112 also present in a keyword database) can be assigned a rank or score or the like with program logic executing one or more algorithms. The ranking or scoring 1118 can be substantially simultaneous with the parsing text 1116 or performing later in time than the parsing text 1116. In at least one embodiment, the keywords database or a related database comprises rank or score data linked to the keywords. In example radiological applications, keywords that denote normal anatomy or lack of findings (normal diagnoses) may be assigned neutral or negative ranks or scores. Keywords that denote normal variants may be assigned low positive ranks or scores, and keywords of abnormal anatomy or pathology may be assigned increasingly higher positive ranks or scores depending upon the seriousness of the finding. For instance, the keywords “lung fields are clear” may be linked to a rank or score of zero or a negative rank or score. The keyword “infiltrate” may be linked to a rank or score of one. The keywords “pleural effusion” may be linked to a rank or score of three. “Subdiaphragmatic air” may be linked to a rank or score of seven. “Pneumothorax” may be linked to a rank or score of six. “Tension Pneumothorax” may be linked to a rank or score of ten. These examples are provided for illustrative purposes only. Of course, alternative scoring algorithms in which increasingly serious diagnoses or interpretations are numerically distinguished from routine or non-serious diagnoses are also contemplated. In some embodiments, very rich data may be assigned a lower rank or score than simple data, which is assigned a higher rank or score. Furthermore, more sophisticated scoring algorithms can also be used.

Certain conditional or relational terms can affect the richness of the interpretation, for example, because the terms change the descriptiveness, amount of detail, seriousness, or criticality of the interpretive output. For instance, interpretive output noting “large calcified nodules,” is richer than a finding of “significant calcified nodules,” which is richer than “no calcified nodules.” Thus, in certain embodiments, certain words in the interpretation 1112 such as conditional or relational terms can be used to provide context 1120 when ranking or scoring of the interpretation 1112. An example algorithm follows. The processor 1110 parses text in the interpretation 1112. The processor 1110 determines whether a word is stored in a conditional or relational term database. The processor 1110 determines whether the word appears adjacent a keyword in the interpretation 1112 or within a defined number of terms of a keyword in the interpretation 1112. Depending on a rank or score assigned to the word in the conditional or relational term database or a database linked thereto, the processor 1110 increases or decreases the score or rank of the keyword if the conditional or relational word is present and adjacent the keyword or within a defined number of terms of the keyword. The foregoing algorithm is provided as an example; alternative scoring algorithms are also contemplated.

In certain embodiments, the requisition 1104 data can be used to provide context 1120 during ranking or scoring of interpretation 1112. For example, keyword parsing of the requisition 1104 data may determine that the requisition 1104 relates to an X-ray or CT scan of the lungs. “Calcified Nodule” in the context of the lungs may be assigned a rank or score of three, but in the context of a mammogram may be assigned a rank or score of eight. An example algorithm follows. The processor 1110 parses text in the requisition 1104 data. The processor 1110 determines whether a word matches a keyword stored in a database. The database can be the same as the database described above in the relation to interpretation 1112, or it can be a different database, depending upon the application. The processor 1110 increases or decreases the score or rank of the keyword depending on the presence or absence of a requisition word. The foregoing algorithm is provided as an example; alternative scoring algorithms are also contemplated.

As detailed above, when ranking or scoring keywords 1118 in the interpretation 1112, context 1130 can be provided from the interpretation 1112, and/or context 1132 can be provided from the requisition 1104 data, as desired.

As shown in box 1122, the processor can generate one or more cumulative ranks or scores for the interpretation 1112. A cumulative rank or score 1122 reflects a rank or score of one set of interpretation 1112. In certain embodiments, a cumulative rank or score 1122 can be related to the arithmetic sum, the average, or some other accumulation of the ranks or scores of the keywords in the interpretation 1112, adjusted for context 1130 from the interpretation 1112 and/or adjusted for context 1132 from the requisition 1104 data, when desired.

As discussed later in this disclosure, the processor can also optionally aggregate the cumulative ranks or scores of interpretation 1112. An aggregated rank or score reflects a rank or score of multiple sets of interpretation 1112. In certain embodiments, the aggregate score or rank can be related to the arithmetic sum, the moving average, or some other aggregation of the interpreter's 1108 cumulative ranks or scores 1122. The aggregate score or rank can track or otherwise reflect whether the interpreter 1108 is generating rich interpretation 1112 over time.

In certain embodiments, the system can optionally rank or score keywords in the requisition 1104 (that is, the words in the requisition 1104 also present in a keyword database). This ranking or scoring can, for example, indicate the criticality of the requisition 1104. For example, the keywords “rule out” can be linked to a rank or score of four. The keywords “history of” can be linked to a rank or score of seven. Similarly, the appearance of a significant diagnosis, such as “status-postmyocardial infarction” can have a significantly higher rank or score (e.g., a rank or score of eight or nine) than “pre-op exam” or “screening or preventative health examination” (e.g., a rank or score of one or two). Optionally, the keywords of the requisition 1104 can be adjusted for context 1134, to reflect the richness of the requisition 1104, using the methods described above. For instance, a requisition requesting an interpreter to “rule out recurrent,” is richer than a requisition to “rule out.” Thus, in certain embodiments, certain words in the requisition 1104 data such as conditional or relational terms can be used to provide context 1134 when ranking or scoring the requisition 1104 data.

As shown in box 1126, the processor can optionally generate a cumulative rank or score for the requisition 1104 data. A cumulative rank or score 1126 reflects a rank or score of a requisition 1104. The cumulative rank or score 1126 can be related to the arithmetic sum, the average, or some other accumulation or aggregation of the ranks or scores of the keywords in the requisition 1104 data.

Optionally, the processor 1110 can compare the richness of the interpretation 1112 and the richness of the requisition 1104 data. For example, as shown in box 1128, the processor 1110 can compare the respective cumulative rank or score of the interpretation 1112 (box 1122) and the cumulative rank or score of the requisition 1104 data (box 1126).

As shown in box 1130, the system (for example, the processor 1110) can determine whether the cumulative rank or score of the interpretation 1112 is deficient with respect to the cumulative rank or score of the requisition 1104 data. For example, the processor 1110 can determine whether the cumulative rank or score of the interpretation 1112 meets a threshold value, such as a value equal to the cumulative rank or score of the requisition 1104 data. As another example, the processor 1110 can determine whether the cumulative rank or score of the interpretation 1112 falls within a range from the cumulative rank or score of the requisition 1104 data (e.g., within a given percentage of the cumulative rank or score of the requisition 1104 data, or within a given number from the cumulative rank or score of the requisition 1104 data).

If the interpretive rank or score is deficient, that is, if the cumulative rank or score of the interpretation 1112 does not meet the threshold, the interpreter 1108 can be automatically notified to provide richer interpretation 1112, as shown in line 1131. For example, the notification can include automatically transmitting to the interpreter 1108 an electronic communication regarding the deficiency. The system can also automatically resend the file(s) 1106 to the interpreter 1108 and, with the file(s) 1106, provide an instruction or notation that additional richness in the interpretation is required. The electronic communication can be transmitted to, for example, a computer, a workstation, a PDA, a smart phone, or another computing device accessible by the interpreter 1108. Other suitable means for notifying include voicemail, paging, text messaging, and equivalents thereof.

If the interpretive rank or score is not deficient, that is, if the cumulative rank or score of the interpretation 1112 meets the threshold value, then the system make take one or more actions 1132 directed to the interpreter 1108, the requester 1102, or both.

One example action 1132 comprises automatically sending prescreened data files 1106 to the interpreter 1108. Various embodiments include the realization that workflow processing can be improved by inserting prescreened data into the workflow. Nevertheless, workflow processing does not necessarily include inserting prescreened data in all embodiments. “Prescreened” is a broad term and is to be given its ordinary and customary meaning to a person of ordinary skill in the art (that is, it is not to be limited to a special or customized meaning). Data can be considered prescreened if they have been previously reviewed and identified as having certain characteristics by a reviewing entity other than the interpreter 1108. Prescreening can take many forms, depending on the workflow. For example, prescreening can be a peer review's consensus interpretation of a patient's medical image file, such as normal, abnormal, cancerous, pre-cancerous, benign, unknown, or uncertain, etc. Prescreening can be a technician's disposition of a semiconductor wafer after photolithography processing, such as normal, closed contacts, pattern lifting, etc. Prescreening can be an evaluation of luggage X-rays, such as safe or unsafe. Prescreening can be a recommendation for an action such as “do not replace the part,” “maintain the current temperature,” “turn 90° left,” “do not collect a sample here,” etc., depending on the particular workflow. Prescreening can be a numerical, textual, binary, Boolean value, or the like.

The interpreter's interpretation 1112 generated from prescreened data files 1106 can be compared with the correct interpretation, enabling the interpreter's interpretive output to be monitored and improved. This concept is described in greater detail later in this disclosure.

Moreover, according to the aggregated rank or score of the interpreter 1108, more or less prescreened data files 1106, or richer or less rich prescreened data files 1106 can be presented to the interpreter 1108. For example, a low aggregated rank or score may indicate that the interpreter 1108 is not generating rich interpretations 1112. In such case, very rich prescreened data files 1106 can be presented to the interpreter 1108. The very rich prescreened data files 1106 may improve workflow processing by giving the interpreter 1108 more interesting or challenging data to review. In addition, as described later in the disclosure, the interpretation 1112 based on the prescreened data files 1106 can be compared with the correct interpretation to help identify whether the low aggregated rank or score may be attributable to interpreter 1108 error or oversight.

In addition, or alternatively, according to the aggregated rank or score of the interpreter 1108, files associated with a rich requisition 1104 may be presented to the interpreter 1108. For example, a low aggregated rank or score may indicate that the interpreter is not receiving file(s) 1106 with rich data or is receiving a small number of file(s) 1106. Presenting the interpreter 1108 with a rich requisition 1104 may improve workflow processing by giving the interpreter 1108 interesting or challenging data to review or by directing rich file(s) to an interpreter 1108 with a higher capacity to handle the file(s). In addition, if a requisition is of a sufficiently high rank, it might be presented for multiple interpretations to several interpreters.

In addition, or alternatively, if the aggregated rank or score of the interpreter does not meet a threshold value or falls below a threshold value, the interpreter 1108 can be notified via an electronic communication. The notification may comprise an electronic message instructing the interpreter 1108 to take a break, a pop-up game displayed on the workstation of the interpreter 1108, or a system lock-out, for example. This concept is also described in greater detail later in this disclosure.

Another suitable action 1132, alone or in combination with the above-described action or any other actions described herein, comprises automatically transmitting, to the requester 1102, an electronic communication concerning the interpretation 1112. The electronic communication can be transmitted to, for example, a computer, a workstation, a PDA, a smart phone, or another computing device accessible by the requester 1102. In some embodiments, the electronic communication is triggered by a condition concerning the interpretation 1112. For example, the electronic communication can be triggered when the cumulative rank or score 1122 of the interpretation 1112 exceeds a threshold value. As another example, the electronic communication can be triggered when, during parsing the text in the interpretation for keywords (box 1116), the parsing detects a given keyword, such as a keyword indicating a critical condition. In some embodiments, the electronic communication can be sent on an urgent basis, for example, by text voicemail, paging, text messaging, and equivalents thereof, which can provide the requester 1102 with the notification in substantially real time. Real time notification can be desirable to communicate an abnormal or critical condition as quickly as possible. This concept is also described in greater detail later in this disclosure.

Another suitable action 1132, alone or in combination other actions, can involve an operation based on the cumulative rank or score 1126 of the requisition 1104. For instance, if the processor 1110 determines that the cumulative rank or score 1126 for requisition 1104 is sufficiently rich, for example, because the cumulative rank or score 1126 exceeds a threshold, the system can electronically mark the associated file(s) 1106 for distribution to multiple interpreters 1108. Using this strategy, it is possible to automatically trigger a consensus interpretation of potentially difficult or otherwise rich data in file(s) 1106. Other suitable actions, such as adding the file(s) 1106 to a file library and/or central repository or sending the file(s) to a case review committee or a peer review committee, are described later in this disclosure.

Additional suitable actions 1132, alone or in combination with the above-described action or any other actions described herein, can involve entities other than the interpreter 1108 and the requester 1102, as shown by line 1134. For instance, if the processor 1110 determines that the cumulative rank or score 1122 for interpretation 1112 is sufficiently rich, for example, because the cumulative rank or score 1122 exceeds a threshold, the system can electronically mark the associated file(s) 1106 for distribution to another interpreter 1108 or a group of interpreters 1108. Using this strategy, it is possible to automatically trigger a consensus interpretation of difficult or otherwise rich data in file(s) 1106. Other suitable actions, such as adding the file(s) 1106 to a file library and/or central repository or sending the file(s) to a case review committee or a peer review committee, are described later in this disclosure.

In some embodiments, the action 1132 is triggered after the system determines whether the rank or score is deficient (box 1130). Nevertheless, as shown in line 1136, the system can take an appropriate action 1132 without first determining whether the rank or score is deficient (box 1130).Many types of data network connections are suitable for interconnecting components described herein. Suitable data network connections can be, for example, a local area network (LAN) connection, wide area network (WAN) connection, wireless local area connection (WLAN), the Internet, virtual private network (VPN) connection, secure sockets layer (SSL) connection, or the like. Additionally, a data network connection can be an electronic connection between a keyboard and a computer, between a media reader and a computer, between hardware components in a computer (e.g., a bus connection between a hard drive and module), or the like. References to “transmitting,” “communicating,” “sending,” and the like as used in this description refer to delivering data over a suitable data network connection.

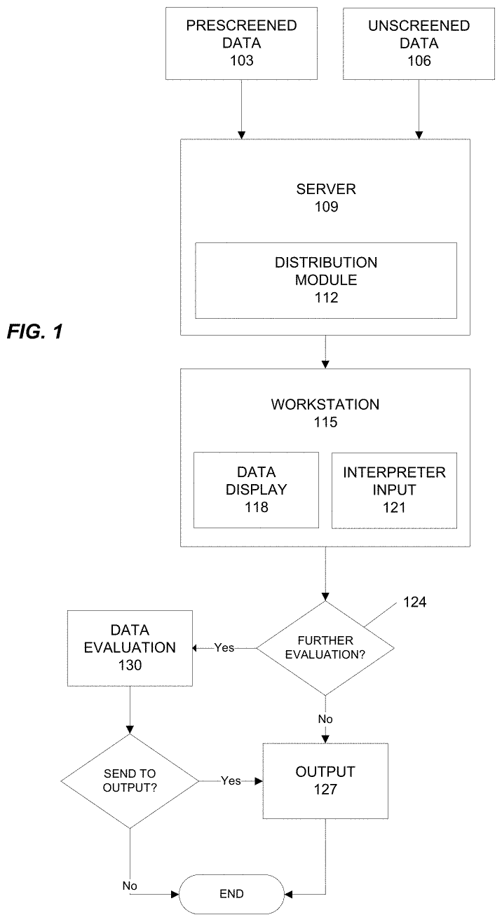

For a more detailed understanding of the disclosure, reference is next made to FIG. 1, which illustrates a workflow system according to at least one embodiment. The system comprises, optionally, a source of prescreened data 103 and a source of unscreened data 106. The prescreened data 103 and unscreened data 106 can be transmitted to at least one interpreter workstation 115 through a server 109. The data processed at the workstation 115 can optionally be transmitted to a decision module 124 to determine whether the data requires further evaluation. Thereafter, the data can be transmitted to an output 127 and/or a data evaluation 130 module.

As previously discussed, “prescreening” can refer to identifying certain characteristics of data before data is intentionally inserted for later review by an interpreter. In contrast, unscreened data 106 is data that has not previously been characterized. “Unscreened” is also a broad term and is to be given its ordinary and customary meaning to a person of ordinary skill in the art (that is, it is not to be limited to a special or customized meaning). An example of unscreened data 106 is production workflow data, such as an undiagnosed DICOM image.

The prescreened data 103 can be transmitted to the server 109 along with the unscreened data 106. In some embodiments, the server 109 can be a central PACS server. The central PACS server can acquire, via a data network connection, unscreened and prescreened DICOM files. Other types of servers that can acquire files from an entity are also suitable. For example, a server can be a central computer on an aircraft that acquires sensor data from a plurality of sub-computers associated with a plurality of sensors via various network connections. The server can comprise a distribution module 112. The distribution module 112 can be configured to distribute the prescreened data 103 and the unscreened data 106 to one or more interpreter workstations 115.

The workstation 115 may include a data display 118 and permit the interpreter to review data. The workstation 115 may also include an interpreter input 121 and permit the interpreter to enter interpretations of data.

An interpreter may be a clinician such as an interpreting radiologist or cardiologist who reviews DICOM files or a photographic data analyst. However, an interpreter may also be a business entity, a semiconductor process technician, a luggage inspector, a manufacturing test diagnostician, a pilot, or the like. As mentioned above with reference to FIG. 1, the workstation may include a data display configured to present data for interpretation, and an interpreter input configured to receive interpretations of data.

The interpretation entered in the interpreter input 121 can optionally be transmitted to a decision module 124. The decision module 124 may determine if the evaluated workflow data was prescreened or unscreened workflow data. If the workflow data was prescreened, then the interpretation may be transmitted to a data evaluation module 130, described in more detail below. If the workflow data was unscreened, then the interpretation may be sent to the system output 127. However, as discussed in more detail below, the decision module 124 can evaluate other data characteristics.

System output 127 can be any entity, server, computing device, network of computing devices, storage system (e.g., a backup system or database), display system (e.g. a monitor or printer), workflow or the like that receives interpreters' interpretations of workflow data. System output can be in the same workflow or in a different workflow. Many types of configurations are suitable for system output. For example, system output can be a CIS for a healthcare provider or pool of healthcare providers. In this example, the CIS can receive diagnoses based upon production workflow medical images from interpreters (e.g. clinicians or radiologists) workstations, and the interpretation is then examined by a physician (e.g. a treating physician). As another example, system output can be a computer onboard an airplane that controls certain flight equipment. The onboard computer can receive a pilot's interpretation of certain sensor readings that the pilot communicates via a keypad or via a speech-interpreting unit.

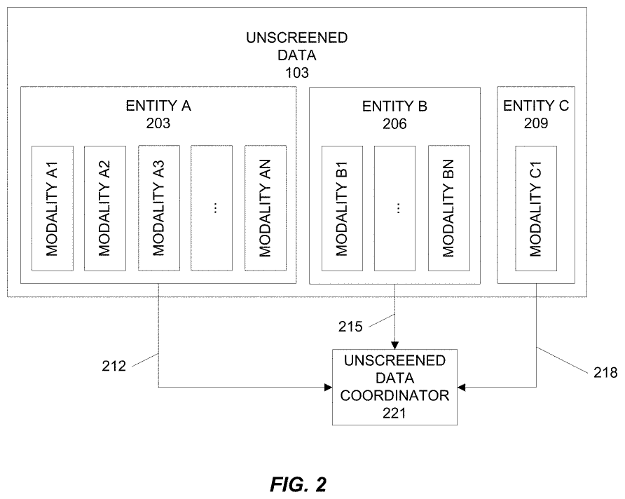

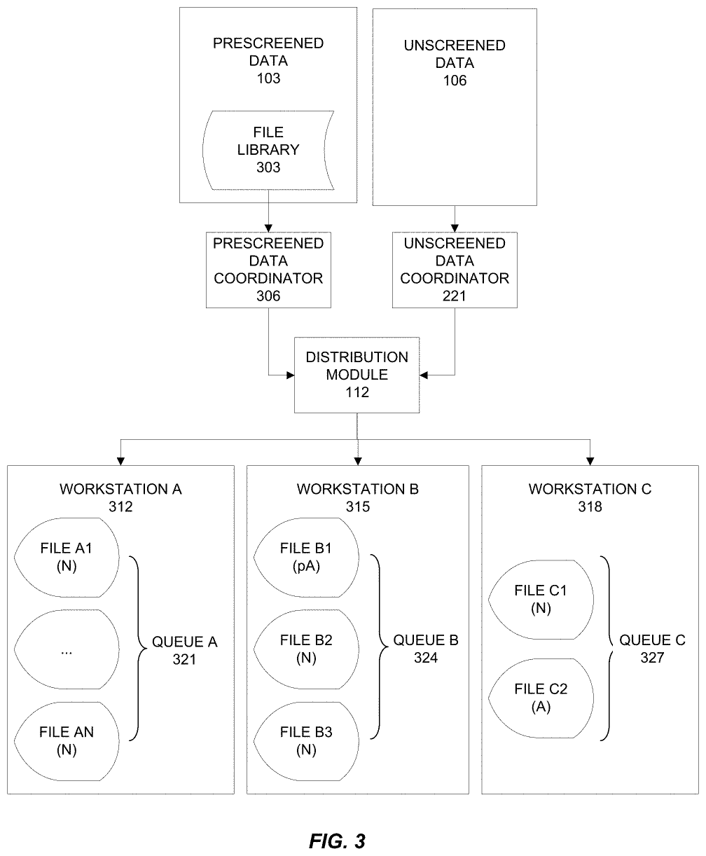

FIG. 2 illustrates sources of unscreened data 103, for example, production workflow data. Entity A 203 and Entity B 206 each comprise a plurality of modalities that produce workflow data. Entity C 209 comprises a single modality. Modalities can be associated with an entity via a suitable connection, such as a data network connection, an electronic connection, a virtual connection, or the like. The entities 203, 206, 209 may transmit production workflow data to an optional unscreened data coordinator 221 (discussed in more detail with respect to FIG. 3) via data network connections 212, 215, and 218.

The entities shown in FIG. 2 (Entity A 203, Entity B 206, and Entity C 209) can be healthcare entities such as hospitals, clinics, imaging centers, medical offices, physicians, or the like. An entity can also be a corporation, airport terminal, semiconductor fabrication laboratory, an assembly line, business unit, network of computers, a single computer, or the like.

In some embodiments, an entity processes the data received from a modality. For example, the entity can format, modify, compress, delete, and/or store data received from a modality. In some embodiments, the entity does not process the data the received from a modality. For example, the entity can be a “virtual entity” that transmits data received from a modality without performing any other processing of the data.

Modalities can be medical imaging devices that generate DICOM files, such CT scanners, MRI scanners, X-ray scanners, DCRs, mammography machines, fluoroscopy machines, etc. A modality can be a video camera, wafer prober, thermocouple, or sensor such as a light, speed, voltage, or altitude sensor. A modality can be a media reader such as a ZIP, floppy, CD, DVD, or other computer drives that reads electronic files stored on computer-readable media. As another example, a modality can be a keypad for entering information into files.

A modality creates “unscreened” data, such as production workflow data. Unscreened data has not been identified as having certain characteristics before the data is inserted into a workflow for a later review by an interpreter. For example, a DICOM file can be considered unscreened if a diagnostic clinician has not yet reviewed the images embedded in the file. A patterned semiconductor wafer can be considered unscreened if the wafer has not yet been reviewed by a technician to identify and/or characterize pattern defects. A piece of luggage can be considered unscreened if the luggage has not yet passed to a TSA screener for evaluation.

Workflow data (that is, unscreened and/or prescreened data) can be binary-format files such as image or sound files or formatted text files. For example, in some embodiments, the data is a DICOM-format file. A DICOM (Digital Imaging and Communications in Medicine) file comprises medical examination images as well as patient and examination information such as patient demographics, patient name, patient age, exam number, exam modality, exam machine name, and exam date in a file header. Data can also be ASCII files such as unformatted text files. Data can also be tangible objects. For example, a photolithocell modality may generate patterned semiconductor wafers.

FIG. 3 illustrates an example embodiment comprising prescreened data 103 obtained from a file library 303. FIG. 3 also illustrates operation of the workflow distribution module 112, the prescreened data coordinator 306, and the unscreened data coordinator 221.

In some embodiments, the source of prescreened data 103 comprises one or more file libraries 303. A file library 303 is configured to send prescreened data. A file library 303 can be a database or files on a computer-readable medium such as a ZIP disc, external hard drive, CD, DVD, or the like. A file library can be local or stored on a remote network or server. For example, a file library can comprise prescreened digitized images, X-rays, HL-7 compliant files, etc.; files prescreened by a third-party provider such as a CME (continuing medical education) or other continuing education program, a commercial vendor, a peer review board or program, an Internet web site; or files prescreened by entities comprised by the workflow (for example, a physician from a hospital entity can have certain patient images stored locally on a personal computer).

A file library 303 can be implemented in conjunction with an optional prescreened data coordinator 306. The prescreened data coordinator 306 can communicate with the file library 303, for example, via a data network or other suitable connection. The prescreened data coordinator 306 may be located on the same computer that stores the file library 303 and/or the distribution module 112. Alternatively, the prescreened data coordinator 306 can be a separate local or remote computer.

In certain embodiments, the prescreened data coordinator 306 is configured to coordinate the insertion of prescreened data from the file library 303 into a distribution module 112. The prescreened data coordinator 306 can comprise human decision-makers, such as a case review committee or a peer review committee. Preferably, the prescreened data coordinator 306 comprises a computer system that manages the input and output of data. The prescreened data coordinator 306 can be located on the same computer as the file library 303 and/or distribution module 112 or on a different local or remote computer. Adjustments of the amount of prescreened data to be presented to interpreters may be performed by the prescreened data coordinator 306, and the adjustment may then be transmitted to a distribution module 112. This may result in a centralized system-wide adjustment, and may be performed remotely.

In some embodiments, the prescreened data coordinator 306 can access, select, and/or electronically mark, flag, or otherwise alter data or files obtained from the file library 303 for subsequent distribution by the distribution module 112. Many types of electronic marks are suitable, such as setting a Boolean or binary switch or flag in a file, inserting a switch or flag the file's header (e.g., in a DICOM file header), altering the file name, inserting a particular string, switch, or flag within the file, or the like. In various embodiments, the prescreened data coordinator 306 can move the altered file outside the file library, or the prescreened data coordinator 306 can copy the original file and store the altered file outside the file library.

For instance, a human prescreened data coordinator 306 using a general purpose computer can access a DICOM files stored in a file library 306. The prescreened data coordinator 306 can select a DICOM file and alter the data in the DICOM file header to indicate how the file will be subsequently distributed by a distribution module 112. The distribution module 112 accordingly can be configured to access the DICOM file header to look for certain altered data for distribution instructions.

In some embodiments, the prescreened data coordinator 306 can comprise a database configured to store information about a file in the file library 303, which information can be accessed by the distribution module 112. For example, the prescreened data coordinator 306 can process a DICOM file and access a database table associated with the DICOM file. The data in the table can be altered to indicate how the file will be subsequently distributed by the distribution module 112. For example, the table can be configured to store the file name and information regarding to which interpreter login that file is to be distributed. The distribution module 112 accordingly can be configured to access the table to look for distribution instructions.

In the example shown in FIG. 3, the prescreened data coordinator 306 can mark a file from the file library 303 such that the distribution module 112 will distribute the file to workstation A 312, workstation B 315, and workstation C 318. In another instance, the prescreened data coordinator 306 can electronically mark a file so that the file is distributed only to workstation A 312. The prescreened data coordinator 306 can control a variety of parameters, for example, location, time, order, etc. of the files inserted.

In some embodiments, the prescreened data coordinator 306 marks files according to a predefined set of constraints. For instance, the prescreened data coordinator 306 can mark data such that one file will be distributed to each workstation twice per hour. In some embodiments, the prescreened data coordinator 306 can mark files according to dynamically received and learned events. Dynamic and learned processing will be discussed in more detail below.

The optional unscreened data coordinator 221 can be configured similar to the above-described prescreened data coordinator 306. The unscreened data coordinator 221 can communicate with the source of unscreened data 106, for example, via a data network or other suitable connection. The unscreened data coordinator 221 stores information about unscreened data 106, such as originating entity and or modality, patient ID, primary-care physician, number and location of related images, number and/or identity of diagnostic physicians. The unscreened data coordinator 221 may be located on the same computer that stores the unscreened data 106 and/or the distribution module 112. Alternatively, the unscreened data coordinator 221 can be configured on a separate local or remote computer. In various embodiments, the prescreened data coordinator 221 can be configured to store unscreened data. However, the prescreened data coordinator 221 can also be configured to point to remotely stored unscreened data.

In certain embodiments, the unscreened data coordinator 221 is configured to coordinate the insertion of unscreened data 106 into a distribution module 112. The unscreened data coordinator 221 can comprise human decision-makers, such as primary-care physicians. Preferably, the prescreened data coordinator 221 comprises a computer system that manages the input and output of data.

For instance, a human unscreened data coordinator 221 using a general purpose computer can access a DICOM file stored in a source of unscreened data 106. The unscreened data coordinator 221 can select the DICOM file and alter the data in the DICOM file header to indicate how the file will be subsequently distributed by a distribution module 112. The distribution module 112 accordingly can be configured to access the DICOM file header to look for certain altered data for distribution instructions. In other embodiments, the unscreened data coordinator 221 can comprise a database configured to store information about an unscreened data 106 file, which information can be accessed by the distribution module 112. For example, the unscreened data coordinator 221 can access a database table associated with the DICOM file. The data in the table can be altered to indicate how the file will be subsequently distributed by the distribution module 112. For example, the table can be configured to store information regarding how many workstations the file should be distributed to. The distribution module 112 accordingly can be configured to access the table to look for distribution instructions.

The prescreened data coordinator 306 and/or the unscreened data coordinator 112 can be omitted in various embodiments. For example, the files stored in a file library 303 can be transmitted to a distribution module 112 automatically as part of a subscription service. As another example, unscreened data 106 does not need to have its attributes modified. As further example, the distribution module 112 may be configured to control and/or process files stored in the file library, by using the mechanisms explained above. Alternatively, the file library 303 may be configured to control and/or process stored files.

As explained above, a distribution module 112 can acquire unscreened data 106 (such as production workflow data) and prescreened data 103. The distribution module 112 distributes, via suitable data network connections, unscreened data 106 and prescreened data 103 to one or more workstations 312, 315, 318 for processing. The distribution module 112 can comprise a computer performing a preprogrammed or dynamic calculation. In some embodiments, the distribution module 112 can comprise a human performing a manual calculation.

The distribution module 112 can operate as a sequential module that retrieves a file upon a request by a workstation. In some embodiments, the one or more workstations 312, 315, 318 can send a request for a file to the distribution module 112. The distribution module can then request the file from a source of unscreened data 106 or the source of prescreened data 103 and send the file to the requesting workstation. The requesting workstation can also request the file from the source of prescreened or unscreened data directly without intermediate processing by the distribution module 112. In some embodiments, the distribution module 112 can select the file from a pool of files comprising unscreened data 106 and prescreened data 103 randomly or according to one or more selection criteria. These selection criteria are described in more detail in this disclosure.

Various embodiments include the realization that processing speed can be improved by creating file queues. As discussed in more detail below, a file queue can be assembled independent of an interpreter's requests for a file. Preferably, a file queue is assembled prior to an interpreter's request for a file. A queue can comprise one or more data items, such as data files, to be processed by an interpreter. A queue can comprise, for example, one, tens, hundreds, or thousands of data items. Preferably, a queue comprises a plurality of data items. An example queue comprises a queue of DICOM files. Queues created and distributed to interpreter workstations need not comprise an identical number of data items. For example, in FIG. 3, queue A 321 comprises N files, queue B 324 comprises three files, and queue C 327 comprises two files. The file queue can be stored on the distribution module 112 or other suitable sever for access by one or more workstations. In some embodiments, the file queue can be transmitted to one or more workstations.

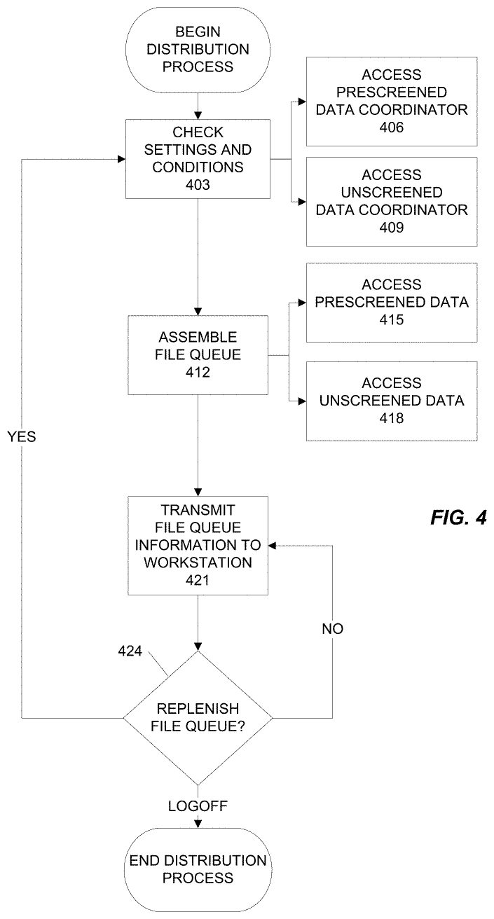

An example distribution process decision tree 400 comprising assembling a single interpreter's file queue is shown in FIG. 4.

The distribution module can optionally check settings and conditions 403. For example, the distribution process can check interpreter and process settings, such as time, date, interpreter login information, workstation ID, etc. As another example, an interpreter with administrative or other certain privileges could set a process setting to temporarily or permanently halt the transmission of prescreened data to a single interpreter workstation, to a group of interpreter workstations, or to all interpreter workstations. The process setting could be set locally or by SSL (interne or intranet), by e-mail, instant message, SMS text message, and the like. Of course, additional interpreter, process, and other settings can be useful determining how and/or when to distribute files to workstations and are contemplated for use in the disclosed embodiments.

In some embodiments, the distribution module can check systemic conditions (such as interpreter file load, average processing time, number or rate of incoming unscreened cases, etc.) For example, a systemic condition could be that the backlog of unscreened files not yet interpreted doubles in one hour. Other systemic conditions could be, for example, that one interpreter's queue is 25% shorter than average, one interpreter's queue is replenishing 50% slower than average, one interpreter's queue is 75% shorter than other interpreters' queues, an interpreter has made more than three incorrect interpretations in the last month, etc. Certain embodiments include the realization that detecting systemic conditions could advantageously avoid overburdening interpreters with prescreened data in the midst of a natural disaster, epidemic, terrorist attack, war, or other event that may create large volumes of production workflow data.

As explained above, the distribution module can also optionally access the prescreened data coordinator 406 and/or the unscreened data coordinator 409 for distribution information. For example, as discussed above, the prescreened data coordinator 406 and the unscreened data coordinator 409 can store information regarding which files in the file library are to be distributed to which interpreter(s), at which workstation(s), at which time(s) or date(s).

Using the settings and conditions information, the prescreened data coordinator information, and/or the unscreened data coordinator information, the distribution module 112 can assemble the file queue 412 utilizing a file assembly algorithm. A variety of algorithms are suitable for assembling the file queue. For example, the file assembly algorithm can insert unscreened data and prescreened data at fixed intervals or random intervals.

Preferably, however, the file assembly algorithm is configured to adjust the amount of prescreened data inserted. For instance, the file assembly algorithm can insert more or less prescreened data into a particular interpreter's queue if it determines or is informed that a certain condition or conditions are met, such as a particularly light or heavy file load, more or fewer errors than other interpreters, etc. As discussed above, the file assembly algorithm can evaluate systemic settings or conditions indicating rapidly increasing backlogs in the number of unscreened files to be processed. Accordingly, the file assembly algorithm can temporarily or permanently halt the transmission of prescreened data to interpreter workstations.

In certain embodiments, the file assembly algorithm may adjust the amount of prescreened data inserted into the workflow dynamically according to received and/or learned events, such as the richness of an interpreter's interpretive output. This feature is discussed in more detail below.

During or after the process of assembling the file queue 409, the distribution module can access the prescreened data 415 and the unscreened data 418. For example, the distribution module can establish links or pointers to prescreened and/or unscreened files stored remotely. Alternatively, the distribution module can download and store files from the sources of prescreened data and unscreened data. The file queue is then ready for access by the interpreter, and the information (files or pointers) in the file queue can be transmitted to a workstation 421.

Still referring to FIG. 4, there are a variety of suitable methods for transmitting the file queue information to a workstation 421. For example, an interpreter can request a file from the distribution module, and the distribution module can send the next file in the file queue to the interpreter. As another example, the distribution process can push all or some of the files in a file queue to an interpreter workstation, and the interpreter workstation can automatically present the next file in a queue.

An optional queue replenishment module 421 can determine whether an interpreter's file queue needs to be replenished. A number of thresholds for the queue decision module 421 are suitable for use herein. For example, a new file queue can be assembled after a previous file queue is pushed to the interpreter's workstation. A new file queue can be assembled when there are a certain number of cases, for example, 10, 5, 1, or 0 cases, remaining to be sent to the interpreter. The queue replenishment module 421 can determine that an interpreter's file queue needs to be replenished at a particular time (e.g., every one or two hours). As another example, the queue replenishment module 421 can listen for a request to create and distribute a new queue. The request could come from an interpreter, a workflow manager or interpreter with administrative or other certain privileges, or the prescreened data coordinator (not shown). The request could be sent, for example, via SSL (interne or intranet), e-mail, instant message, SMS text message, or the like. If the file queue needs to be replenished, then the above-described process begins again. The distribution module can append data files to an existing file queue, or the distribution module can create and distribute a new queue. This process can be repeated until halted or stopped or until an interpreter quits or logs off an interpreter workstation. Of course, even if the process is halted for one interpreter, the process can continue for other interpreters as needed.

Referring again to FIG. 3, the distribution module 112 can be configured to create a desired number of queues. For example, the distribution module 112 can create and/or distribute a file queue to a single interpreter workstation, or the distribution module 112 can create and/or distribute file queues to a plurality of workstations. For example, the distribution module 112 shown in FIG. 3 distributes queue A 321 to workstation A 312, queue B 324 to workstation B 315, and queue C 327 to workstation C 318.

In some embodiments, the same prescreened data file can be distributed to more than one interpreter for interpretation. This can advantageously create a competitive environment in which interpreters compete against each other to make correct interpretations of prescreened data. This can also advantageously avoid inadvertent or intentional collaboration on interpreting prescreened data. In some embodiments, the same unscreened data file can be distributed to more than one interpreter workstation for interpretation. This can advantageously allow a physician to survey multiple diagnostic clinicians for their interpretations of potentially challenging patient data.

An interpreter workstation (e.g., workstation A 312) can be configured to cooperate with a local or remote queue module (not shown). A queue module can be configured to process a file queue (e.g., queue A 321) created and distributed by the distribution module 112. For example, a queue module be configured to serially present files in the queue on a first queued, first presented basis. A queue module can be configured to present files in a queue in other orders, such as randomly, according to certain predefined criteria, or dynamically according to received and/or learned events.

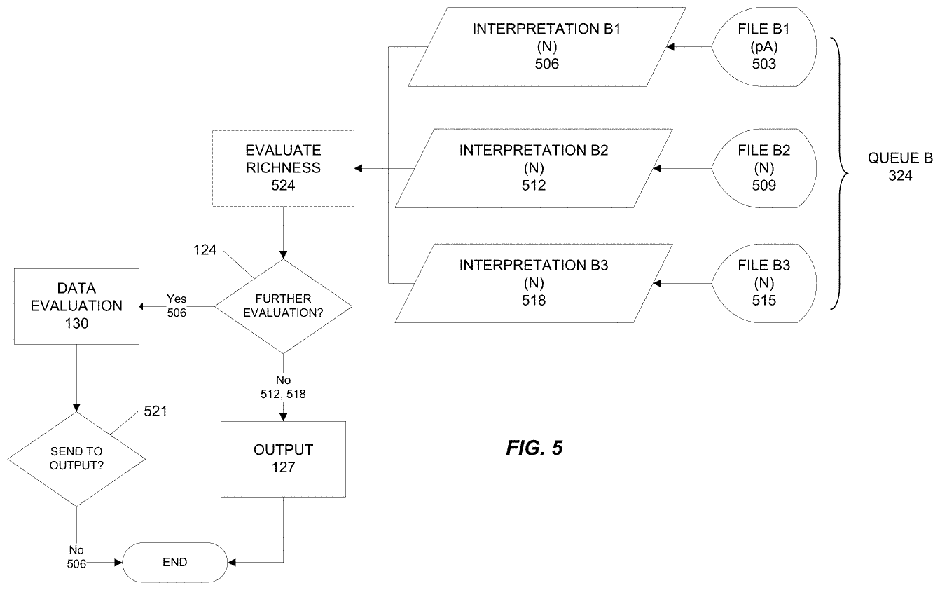

Referring to FIG. 5, an interpreter interprets files (e.g., file B1 503, file B2, 509, and file B3 515) presented on the interpreter workstation (not shown). The interpretations may be sent to system output 127, to a data evaluation module 130, or to both.

An interpreter serially interprets the files in queue B 324. In this example, the interpreter is first presented with file B1 (pA) 503, comprising an image showing a prescreened-abnormal condition.

In various embodiments, the origin of prescreened files can advantageously be “hidden” or “cloaked” from the interpreter, such that the interpreter cannot easily detect whether the file is prescreened or unscreened. Cloaking may be advantageous for training purposes or to improve accuracy and efficiency of data interpretation, as is explained below. Cloaking may be performed centrally, for example, for all files processed by the prescreened data coordinator. In some embodiments, cloaking may be performed on file-by-file basis.

Cloaking may be accomplished, for example, by changing, modifying, spoofing, or otherwise obfuscating file data. DICOM images, for example, comprise a file header (which stores information about the patient's name, the type of scan, image dimensions, etc.), as well two- or three-dimensional image data. In some embodiments, the prescreened data coordinator (discussed above) can be configured to alter information in the data header (such as the study date, origin, etc.), which could alert an interpreter that the data is prescreened.

Alternative cloaking mechanisms are also contemplated for use herein. For instance, cloaking may also be accomplished by masking data displayed on an interpreter's workstation. A workstation processing DICOM files may display images along with textual portions comprising patient information on the data display. In some embodiments, the workstation can be configured to detect whether a file is prescreened and display a text box containing falsified patient data. The text box can overlay the original textual portions in a way that in undetectable by the interpreter. The data in the text may be obtained from the prescreened data coordinator, the data distributor, or from some other source, as needed.

Referring again to FIG. 5, the interpreter (unaware that file B1 503 is not ordinary production workflow data) incorrectly interprets file B1 503 as comprising normal data. The interpreter enters incorrect interpretation B1 (N) 506 at the workstation.

Detecting that the interpretation was based upon prescreened data, the decision module 124 (discussed above with respect to FIG. 1) can direct that the incorrect interpretation B1 (N) 506 be transmitted to a data evaluation module 130 for additional processing. The data evaluation module 130 preferably comprises an output evaluation module 521 configured to determine whether the data should be sent to the system output 127, such as a CIS or HIS. Because file B1 (pA) 503 is not ordinary production workflow data but rather prescreened abnormal data, it may not be desirable to send incorrect interpretation B1 (N) 506 to the system output 130, as shown in FIG. 5. Accordingly, the output evaluation module 521 can evaluate whether file B1 (pA) 503 was flagged as prescreened or otherwise marked for restriction from the system output. Of course, other evaluation methods are also suitable. Furthermore, it may be desirable to send prescreened data to the system output in certain embodiments.

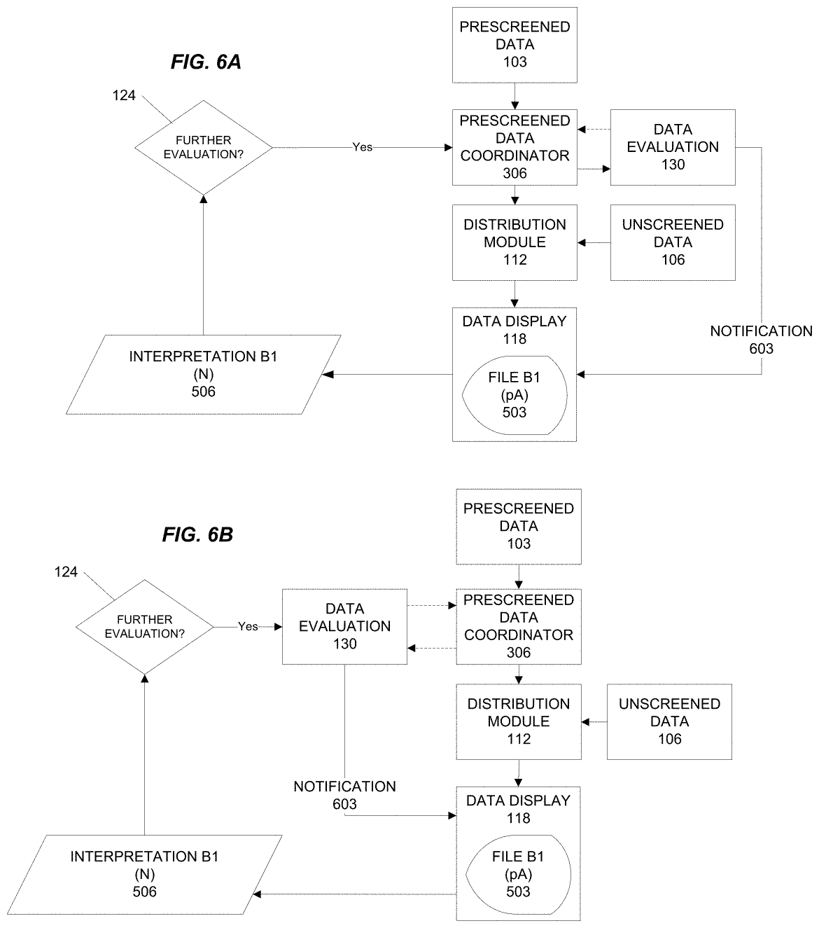

The interpreter may be immediately notified of an incorrect or correct interpretation or notified at a later time. Notification may advantageously help the interpreter to learn and to not make the same or similar mistake again. Immediate notification may help the interpreter to learn in real-time. Example methods for providing notification are shown in FIG. 6A and FIG. 6B. Of course, alternative methods are suitable for use herein and are contemplated by this disclosure.

Referring first to FIG. 6A, a file B1 (pA) 503, comprising a prescreened-abnormal condition, is presented to the interpreter via data display 118. The interpreter incorrectly interprets file B1 503 as showing only normal data. The interpreter enters incorrect interpretation B1 (N) 506 at the workstation. Decision module 124 ascertains that interpretation B1 (N) 506 was based on a prescreened file and determines that further evaluation is needed.

Still referring to FIG. 6A, the interpreter's interpretation B1 (N) 506 may be transmitted to prescreened data coordinator 306. The prescreened data coordinator 306 can access the desired interpretation (abnormal) based on the results of the prescreening and transmit the information to the data evaluation module 130, which is configured to compare the interpreter's interpretation B1 (N) 506 with the desired interpretation.

The prescreened data coordinator 306 can subsequently transmit a notification 603 based on the comparison to the interpreter's data display 118. In some embodiments, the data evaluation module 130 can transmit certain data back to the prescreened data coordinator 306. The data coordinator 306 can store the interpreter's interpretation B1 (N) 506, store the data evaluation module 103 comparison, and/or store whether the interpreter interpretation was correct or incorrect, for example, in an interpreter profile table. This information can be later downloaded by the interpreter's manager and used in the interpreter's performance review or transmitted to the interpreter via notification 603 at a later time (e.g., at the end of an interpreter's shift). In some embodiments, this information can be utilized by the distribution module 112 when deciding whether to display the file to the interpreter at a later time.

An alternative method for providing notification is shown in FIG. 6B. The process is similar to the one described above with respect to FIG. 6A, however, FIG. 6B demonstrates that the prescreened data coordinator 306 is not needed in order to provide notification to an interpreter. For example, the desired interpretation can be embedded in a prescreened file (e.g., in the metadata or in the header data). This embedded data is not ordinarily visible to or easily accessible by the interpreter. However, the embedded data can be displayed to the interpreter, e.g., via a pop-up box or other display mechanism, if the decision module 124 determines that a file was prescreened or otherwise needs further evaluation. As shown in FIG. 6B, however, the interpreter's interpretation is optionally but desirably communicated to the prescreened data coordinator 306. The prescreened data coordinator 306 can retain the data for later review or for distribution instruction purposes.

In some embodiments, the prescreened data coordinator 306 may be used to monitor and the correctness of an interpreter's interpretations of prescreened data. When an interpreter's accuracy falls below a certain percentage or the interpreter makes a certain number of consecutive mistakes, the prescreened data coordinator 306 can take an appropriate action, such as notifying the interpreter to take a break or initiating a puzzle or game for display to the interpreter. In some embodiments, the monitoring notification can be configured to “lock out” the interpreter's access to interpreter input during the break. Monitoring notification can comprise a message or report that may be communicated to the interpreter. Preferably, the monitoring notification is transmitted to the interpreter immediately before any additional files are presented for interpretation. However, the message or report can be delivered after a delay. However, in embodiments that do not comprise a prescreened data coordinator 306, other system elements, such as a server (e.g., server 112 shown in FIG. 1) or distribution module 112, can also perform monitoring.

Injecting prescreened data into a workflow and monitoring interpreters' interpretation of prescreened data may allow enhancing the use of data analysis and data mining capabilities of the workflow. Data analysis and data mining comprise a variety of powerful numerical, statistical, probabilistic, algorithms and methods and the like that operate on data sets. Data analysis and data mining may be used to improve, forecast, and predict interpreters' accuracy and efficiency. For example, an interpreter's accuracy and efficiency can be increased by creating a working schedule that decreases mistakes due to fatigue, such as scheduling periodic breaks to maintain a desired level of interpretational accuracy. An interpreter may make few mistakes when processing data early in the shift, and may require infrequent breaks to maintain a desired level of interpretational accuracy. By monitoring an interpreter's accuracy over the course of a workday, the frequency and length of breaks may be increased to maintain a desired level of interpretational accuracy. As another example, a interpreter's accuracy and efficiency may be improved by scheduling periodic training, and the training can be further custom tailored to the interpreter's needs.

Referring again to FIG. 5, when the interpreter is finished interpreting file B1 (pA) 503, the interpreter is next presented with file B2 (N) 509, an unscreened file comprising normal data. The interpreter correctly enters interpretation B2 (N) 512, which is subsequently sent to system output 127. This process is repeated for file B3 (N) 518.

As shown in FIG. 5, in some embodiments, the richness of an interpreter's interpretation can be evaluated and/or monitored, as shown in box 524. As explained in more detail below, interpretive output information may be dynamically monitored and learned, and this advantageously may allow for flexible, real-time, “on-the-fly” adjustments of the amount and/or characteristics of prescreened data presented to interpreters.

An interpreter's interpretation can be assigned a rank or score or the like. Medical images which denote normal anatomy or lack of findings (normal interpretation) may be assigned neutral or negative ranks or scores. Images which denote normal variants may be assigned low positive ranks or scores, and images of abnormal anatomy or pathology may be assigned increasingly higher positive ranks or scores depending upon the seriousness of the finding. For example, “lung fields are clear” may be assigned a rank or score of zero or a negative score. “Infiltrate” may be assigned a rank or score of one. “Pleural Effusion” may be assigned a rank or score of three. “Subdiaphragmatic Air,” a serious sign of a ruptured bowel or stomach, may be assigned a rank or score of seven. “Pneumothorax,” or collapse of the lung, may be assigned a rank or score of six. “Tension Pneumothorax,” a more severe collapse of the lung with actual displacement of the lung, may be assigned a rank or score of ten. “Calcified Nodule” in the context of the lung may be assigned a rank or score of three but in the context of a mammogram may be assigned a rank or score of eight. As another example, “Displaced Fracture” may be assigned a rank or score of three. “Nondisplaced Fracture” may be assigned a rank or score of two. “Foreign Bodies” may be assigned a rank or score of five. “Surgical Sponge” may be assigned a rank or score of ten.

The above examples are provided for illustrative purposes only. Of course, alternative scoring systems in which increasingly serious diagnoses or interpretations are numerically distinguished from routine or non-serious diagnoses are also contemplated in this disclosure. In some embodiments, very rich interpretation data may be assigned a lower rank or score than simple data, which is assigned a higher rank or score. Furthermore, more sophisticated scoring algorithms can also be used. A rank or score can be assigned for both prescreened data and unscreened data in certain embodiments. Alternatively, the rank or score can be assigned only for prescreened data or only for unscreened data. The rank or score can be assigned for a subset of any of the above-mentioned data sets as well. The system may also track an aggregated rank or score. The aggregated rank or score can be related to the arithmetic sum, the moving average, or some other aggregation of the interpreter's ranks or scores over time. The aggregated or rank or score can be based only on unscreened data, only on prescreened data, or on both unscreened data and prescreened data. According to the interpreter's aggregated rank or score, more or less prescreened data may be presented to the interpreter. For example, if an interpreter's interpretive output rank or score is high (meaning that the interpreter is interpreting a large volume of rich data), less prescreened data may be presented to the interpreter. A rapidly increasing rank or score may indicate that the interpreter is suddenly interpreting large volumes of rich workflow data, such as during a natural disaster, epidemic, terrorist attack, war, or other event that creates large volumes of rich production workflow data. Accordingly, the system may temporarily stop the distribution of prescreened data to the interpreter. As another example, if an interpreter's aggregated rank or score is high, the interpreter's schedule may be adjusted such that the interpreter should take more frequent breaks, longer breaks, or works shorter shifts to maintain a desired level of interpretive accuracy. As another example, if an interpreter's interpretative rank or score is sufficiently high, a second examination of particularly difficult interpreted data may be needed, and the data may be marked or flagged accordingly, as is described above.

Scoring or ranking of an interpreter's interpretation may in some embodiments trigger or otherwise affect reporting of the output. Because the rank or score can be indicative of the degree of richness the interpretation of unscreened data, it may be desirable to communicate the rank or score and/or the associated interpretation with the “outside world,” such as to an entity, a case review committee, a peer review committee, a government agency, the requester, or the like. In some embodiments, the unscreened data coordinator may communicate the rank or score and/or the interpretation, however, a variety of modules, servers, or modules are suitable for this purpose. The communication may be performed by sending an Internet request, email message, instant message, SMS text message, or the like.

In addition, when the aggregated rank or score falls below a threshold value, the system can take an appropriate action, such as notifying the interpreter to take a break or initiating a puzzle or game for display to the interpreter. In some embodiments, the monitoring notification can be configured to “lock out” the interpreter's access during the break.

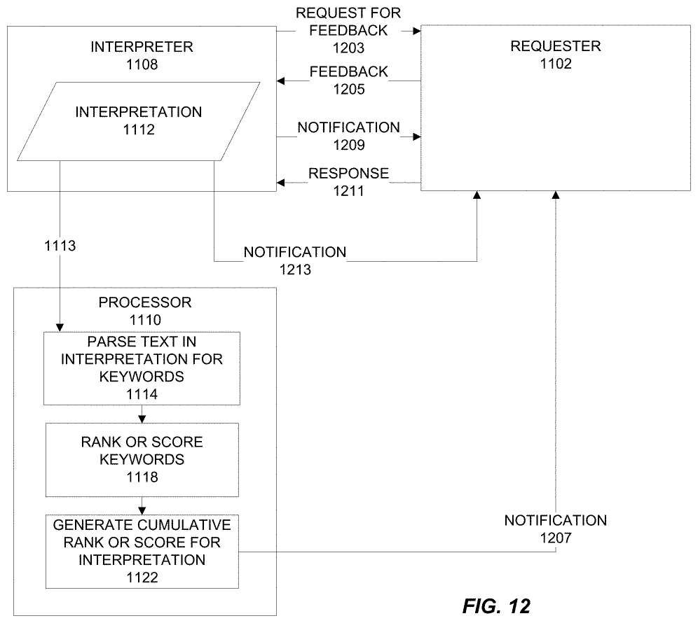

In some embodiments, it may be desirable to solicit or receive feedback relating to an interpreter's interpretation before ranking or scoring the interpretation. Accordingly, various embodiments comprise means for soliciting feedback regarding an interpreter's interpretation. As shown in FIG. 12, an interpreter 1108 can send to a requester 1102 (or other appropriate entity, such as the unscreened data coordinator, the prescreened data coordinator, another interpreter, or an external expert) a request for feedback 1203 regarding a file that the interpreter is interpreting. For example, a radiologist can send to the requesting physician a request for feedback regarding a challenging image that the radiologist is diagnosing. In response to an action from the interpreter 1108, such as selecting an appropriate item from a drop-down menu displayed on the interpreter's display, the system can send the request electronically, e.g., via a request for electronic video conference, instant message, chat request, pop-up notification, e-mail, SMS text message, Internet request, or the like. Preferably, the request is then displayed to the requester 1102 (e.g., on the requester's computer, PDA, smart phone, another computer accessible by the requester's workstation, or another computer accessible by the requester) in real time.

In response to the request, the requester 1102 can send feedback 1205 to the interpreter 1108. For example, the requesting physician can send to the radiologist additional details about the patient's symptoms. In response to an action from the requester 1102, such as selecting an appropriate item from a drop-down menu displayed on the requester's display, the system can send the feedback electronically, e.g., via electronic video conference, instant message, chat, pop-up notification, e-mail, SMS text message, Internet request, or the like. Preferably, the feedback is then displayed to the interpreter 1108 (e.g., on the interpreter's computer, PDA, smart phone, another computer accessible by the interpreter's workstation, or another computer accessible by the interpreter) in real time.

In certain embodiments, the requester 1102 can send feedback 1205 to the interpreter 1108 without first receiving a request for feedback 1203. For example, the requester 1102 may send additional details regarding the patient's symptoms to the interpreter 1108 without an associated request to do so.

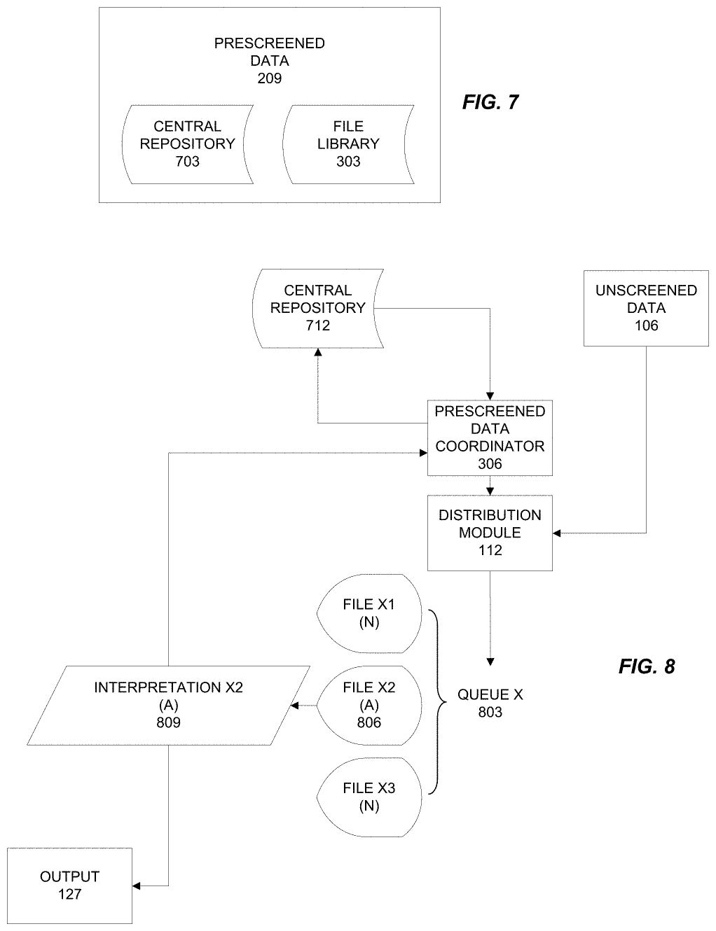

Referring now to FIG. 7, certain embodiments may comprise a central repository 703 as a source of prescreened workflow data. In contrast to a file library 303 (discussed above), a central repository 303 is configured to both send and receive prescreened data. A central repository may be used in conjunction with the file library, or may be used without the file library. FIG. 7 illustrates a central repository 703 of prescreened data 209 in conjunction with a file library 403. A central repository 703 can be located on the same computer as the file library 303 or on a different local or remote computer.

Because a central repository permits both sending and receiving of prescreened data, a central repository 703 can allow different networks to “pool” and/or share their prescreened data together. The central repository 703 may advantageously provide these networks greater access to prescreened data than each network would have on its own. This feature can advantageously allow a network to create a commercially valuable source of prescreened data that can be sold as a file library to other interpreters.

FIG. 8 illustrates how a central repository 703 can be integrated into a workflow. The distribution module 215 receives data from the prescreened data coordinator 406 and the source of unscreened data 106. The data are distributed into queue X 803. The second file in the queue, file X2 (A) 806 comprises abnormal data. The interpreter makes correct interpretation X2 (A) 809, and interpretation X2 (A) 809 is sent to the system output 127.

The interpreter may then flag the file for insertion into the central repository 712. Alternatively, the insertion may take place without any action by the interpreter. Interpretation X2 (A) 809 is sent to the prescreened data coordinator 306. The prescreened data coordinator 306 can transmit file X2 (A) 806 and/or the associated interpretation X2 (A) 809 to the central repository 712.

File X2 (A) 806 can subsequently be used as a prescreened file in a workflow, and its associated interpretation X2 (A) 709 may optionally be used as the desired interpretation for comparison purposes. The central repository 703 can communicate file X2 (A) 806 to the prescreened data coordinator 306. As explained above, the data can subsequently be distributed by a distribution module 112 for presentation to interpreters.

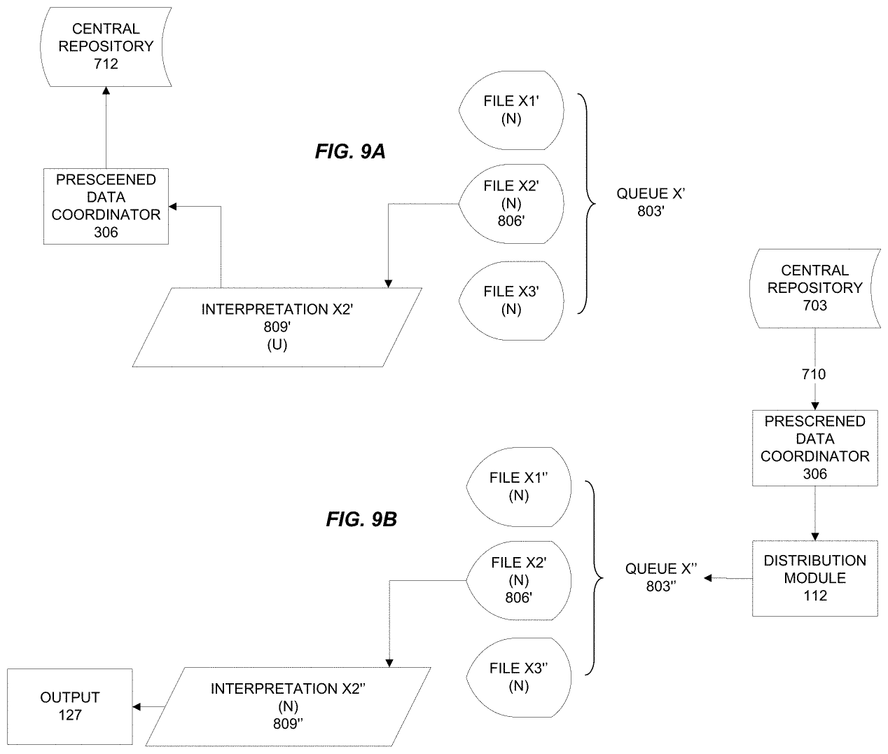

FIG. 9A and FIG. 9B show that the central repository 703 can be useful for facilitating consensus or collaborative diagnoses. An interpreter processes queue X′ 803′. The interpreter is presented with file X2′ (N) 806′, an image showing an unscreened normal condition. In this example, the interpreter is unsure whether file X2′ (N) 806′ comprises normal or abnormal conditions. Accordingly, the interpreter enters that the condition is “unknown” and generates interpretation X2′ (U) 809′ at the workstation. In some embodiments, the interpreter can alternatively or in conjunction mark or flag the file for follow-up interpretation. The file is transmitted via data coordinator 306 to the central repository 712.

Later the file X2′ (N) 806′ is distributed to new queue X″ 803″ by the distribution module 112 after it is received from the central repository 703 by way of the data coordinator 406. Data interpreted as “unknown” may be presented to a new interpreter or the same interpreter for another attempt at the interpretation. In this example, a new interpreter is presented with file X2′ (N) 806′. In this example, the interpreter evaluates the image, correctly decides X2′ (N) 806′ comprises normal data, and generated interpretation X2″ 809″ at the workstation.

The interpretation X2″ 809″ is sent to the system output 127 for subsequent communication to a requester. The image may be untagged as “unknown,” and tagged as diagnosed. Alternatively, the “unknown” tag may be maintained for subsequent interpreters' attempts at the interpretation (e.g., to achieve a consensus interpretation).

In some embodiments it may be advantageous for the interpreter to receive feedback during or after interpretation of prescreened or unscreened data. The workflow interpreter has an interest in ensuring his or her interpretation is received by the requester. The interpreter may also require additional feedback from the requester. Based on the interpreter's interpretation, the requester may provide additional relevant clinical information to the interpreter or the requester may take issue with the final interpretation.

Accordingly, various embodiments comprise means for soliciting feedback regarding an interpreter's interpretation during or after interpretation of prescreened or unscreened data. A suitable technique is discussed above with reference to FIG. 12.

In certain embodiments, the interpreter may want to notify the requester of the findings reported. This can be particularly desirable when the findings show a critical condition that the requester should review on an urgent basis. As shown in FIG. 12, the interpreter 1108 can send a notification 1209 to the requester 1102. Example notifications 1209 include, without limitations, instant message, chat, pop-up notification, e-mail, SMS text message, Internet request, or the like.

In some embodiments, the interpreter 1108 can select a level of criticality for the notification 1209. The level of criticality can also be preset to a given setting. There are various degrees of criticality, such as, without limitation, real-time display of the notification 1209 to the requester 1102 (a lower criticality) and requiring acknowledgment or receipt of the notification 1209 and/or interpretation 1112 by the requester 1102 (a higher criticality). Based upon the criticality, the system may require a response 1211 to be lodged either electronically, by interactive voice response, voicemail, paging, text messaging, direct voice access, and the like. . Based upon the criticality of the findings, a watchdog timer could be established within which time frame a response 1211 would be expected. If the response is not received, the selected means for notification could be escalated to a more direct form of communication to ensure that the findings were timely conveyed.

In some embodiments, the system can notify the requester 1102 via a notification 1207, based on the cumulative score or rank of the interpretation 1122. This is particularly desirable when interpretation 1112 contains critical findings that should be communicated to the requester on an urgent basis. The notification 1207 can be transmitted to, for example, a computer, a workstation, a PDA, a smart phone, or another computing device accessible by the requester 1102. For example, the notification 1207 can be triggered when the cumulative rank or score 1122 of the interpretation 1112 exceeds a threshold value. As another example, the electronic communication can be triggered when, during parsing the text in the interpretation for keywords (box 1116), the parsing detects a given keyword, such as a keyword indicating a critical condition. In some embodiments, the notification 1207 can be sent on an urgent basis, for example, by text voicemail, paging, text messaging, and equivalents thereof, which can provide the requester 1102 with the notification in substantially real time. Real time notification can be desirable to communicate an abnormal or critical condition as quickly as possible. The notification 1207 can be sent to the requester 1102 independent of interpreter intervention.

As mentioned, data presented to the interpreter 1108 may be flagged or marked. In such case, with the methods discussed above, the system can notify the requester 1102 of the interpretation 1112 in substantially real time.

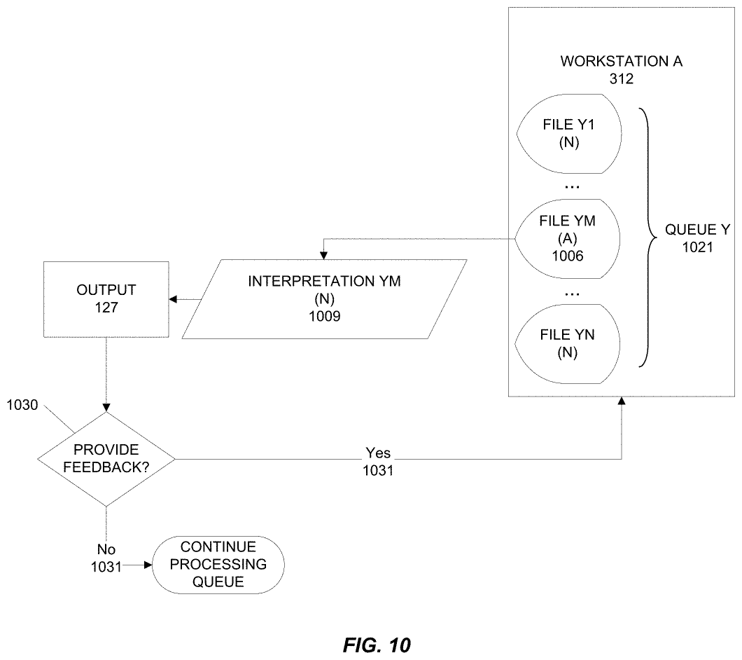

Example methods for providing feedback are also shown in FIG. 10. Of course, alternative methods are suitable for use herein and are contemplated by this disclosure.

As shown in FIG. 10, an interpreter processes queue Y 1021. The interpreter is presented with file YM 1006, and makes an interpretation YM 1009. The interpretation YM 1009 is sent to the system output 127 for subsequent communication to an requester. Subsequently, the interpretation is sent to the feedback module 1030, which can be configured to determine whether the interpretation has been flagged for feedback. The feedback module determines whether the requester needs to provide feedback or whether the queue processing may resume. If feedback needs to be provided, the feedback module determines its type (based on the level of criticality), and provides the appropriate feedback.

Except where indicated otherwise, all of the steps and tasks described herein may be performed and fully automated by a computer system, and may be embodied in software code modules executed by one or more general purpose computers. The code modules may be stored in any type of computer-readable medium or other computer storage device. Some or all of the methods may alternatively be embodied in specialized computer hardware. The computer system may, in some cases, be composed of multiple distinct computers or computing devices (e.g., physical servers, workstations, storage arrays, etc.) that communicate and interoperate over a network to perform the described functions. Each such computing device typically includes a processor (or multiple processors) that executes program instructions stored in a memory or other computer-readable medium. The results of the disclosed methods may be persistently stored by transforming physical storage devices, such as solid state memory chips and/or magnetic disks, into a different state.

The foregoing embodiments make reference to program logic. Such program logic may advantageously be implemented as one or more modules. The modules may advantageously be configured to execute on one or more processors. The modules may comprise, but are not limited to, any of the following: software or hardware components such as software object-oriented software components, class components and task components, processes methods, functions, attributes, procedures, subroutines, segments of program code, drivers, firmware, microcode, circuitry, data, databases, data structures, tables, arrays, or variables. Decision-making may be carried out by a rule-based system, a decision tree, a neural network, a Bayesian or other probabilistic network, a linear-combination or other calculation, or other decision-making system. In the computer-based systems and methods of the embodiments, the computers comprise, by way of example, processors, program logic, or other substrate configurations representing data and instructions, which operate as described herein. The processors can comprise controller circuitry, processor circuitry, processors, general purpose single-chip or multi-chip microprocessors, digital signal processors, embedded microprocessors, microcontrollers, and the like.

A wide variety of variations are possible. Components may be added, removed, or reordered. Different components may be substituted out. The arrangement and configuration may be different. Similarly, processing steps may be added or removed, or reordered. Those skilled in the art will appreciate that the methods and designs described above have additional applications and that the relevant applications are not limited to those specifically recited above. For example, several example embodiments discussed herein are presented in the context of medical treatment. However, the disclosure can be implemented in a wide variety of workflows in many types of industries, e.g., test and manufacturing, business processes, travel, security, etc. Furthermore, it is contemplated that this disclosure relates to virtual workflows, e.g., workflows used in non-production capacities. The interpreters of the system can be, for example, students accessing and processing a simulated workflow via a LAN, WAN, or the Internet. In these embodiments, training data can come from a variety of sources, e.g., a central repository, a file library, and/or modalities. Also, the present invention may be embodied in other specific forms without departing from the essential characteristics as described herein. The embodiments described above are to be considered in all respects as illustrative only and not restrictive in any manner.

Although this invention has been disclosed in the context of certain preferred embodiments and examples, it will be understood by those skilled in the art that the present invention extends beyond the specifically disclosed embodiments to other alternative embodiments and/or uses of the invention and obvious modifications and equivalents thereof. In addition, while several variations of the invention have been shown and described in detail, other modifications, which are within the scope of this invention, will be readily apparent to those of skill in the art based upon this disclosure. It is also contemplated that various combinations or sub-combinations of the specific features and aspects of the embodiments may be made and still fall within the scope of the invention. It should be understood that various features and aspects of the disclosed embodiments can be combined with, or substituted for, one another in order to form varying modes of the disclosed invention. Thus, it is intended that the scope of the present invention herein disclosed should not be limited by the particular disclosed embodiments described above, but should be determined only by a fair reading of the claims that follow.

What is claimed is:

1. A computer-implemented method for processing data comprising:
with a computer system comprising one or more computing devices,
receiving an electronic requisition comprising an order for an interpreter to interpret one or more files;
parsing the electronic requisition to identify requisition words in the requisition;
detecting which of the requisition words or variants thereof is/are present in a first database of keywords;
respectively ranking or scoring the requisition word(s) or variant(s) thereof that is/are present in the first database;
generating a cumulative requisition rank or score based on the rank(s) or score(s) of the requisition word(s) or variant(s);
receiving an electronic interpretation generated by the interpreter, the electronic interpretation comprising interpretive data based on the one or more files;
parsing the electronic interpretation to identify interpretation words in the interpretation;
detecting which of the interpretation words or variants thereof is/are present in a second database of keywords;
respectively ranking or scoring the interpretation word(s) or variant(s) thereof that is/are present in the second database;
generating a cumulative interpretation rank or score based on the rank(s) or score(s) of the interpretation word(s) or variant(s); and
determining whether the cumulative interpretation rank or score meets a threshold of the cumulative requisition rank or score.

2. The method of claim 1, wherein the first database and the second database are the same.

3. The method of claim 1, further comprising notifying the interpreter to modify or supplement the electronic interpretation when the cumulative interpretation rank or score does not meet the threshold.

4. The method of claim 1, further comprising detecting which of the interpretation words or variants thereof are contextual terms and adjusting the rank or score of any interpretation word that is adjacent or within a number of words of a contextual term in the electronic interpretation or the variant thereof.

5. The method of claim 1, further comprising determining a context of the electronic requisition and determining the rank or score of one or more interpretation words or variants thereof based on the context.

6. The method of claim 1, wherein the first database and the second database are computer-processable collections of medical terms.

7. The method of claim 1, wherein respectively ranking or scoring the requisition word(s) or variant(s) thereof occurs substantially simultaneous with detecting which of the requisition words or variants thereof is/are present in the first database of keywords.

8. The method of claim 1, wherein respectively ranking or scoring the interpretation word(s) or variant(s) thereof occurs substantially simultaneous with detecting which of the interpretation words or variants thereof is/are present in the second database of keywords.

9. The method of claim 1, wherein respectively ranking or scoring the interpretation word(s) or variant(s) thereof comprises assigning an increasingly higher rank or score depending on a seriousness, abnormality, or criticality of a condition, finding, or variation indicated by the respective word or variant thereof.

10. A computer system including one or more computer devices, the system comprising:
an requisition receiver configured to receive an electronic requisition comprising an order for an interpreter to interpret one or more files;
a requisition parser configured to identify requisition words in the requisition;
a requisition detector configured to detect which of the requisition words or variants thereof is/are present in a first database of keywords;
a requisition ranking or scoring module configured to respectively rank or score the requisition word(s) or variant(s) thereof that is/are present in the first database;
a cumulative requisition ranking or scoring module configured to determine a cumulative rank or score for the electronic requisition based on the rank(s) or score(s) of the requisition word(s) or variant(s);
an interpretation receiver configured to an electronic interpretation generated by the interpreter, the electronic interpretation comprising interpretive data based on the one or more files;
an interpretation parser configured to parse the electronic interpretation to identify interpretation words in the interpretation;
an interpretation detector configured to detect which of the interpretation words or variants thereof is/are present in a second database of keywords;
an interpretation ranking or scoring module configured to respectively rank or score the interpretation word(s) or variant(s) thereof that is/are present in the second database;
a cumulative interpretation ranking or scoring module configured to generate a cumulative interpretation rank or score based on the rank(s) or score(s) of the interpretation word(s) or variant(s); and
a determiner configured to determine whether the cumulative interpretation rank or score meets a threshold of the cumulative requisition rank or score.

11. The system of claim 10, wherein the first database and the second database are the same.

12. The system of claim 10, further comprising a notifier configured to notify the interpreter to modify or supplement the electronic interpretation when the cumulative interpretation rank or score does not meet the threshold.

13. The system of claim 10, further comprising a first context adjuster configured to detect which of the interpretation words or variants thereof are contextual terms and adjust the rank or score of any interpretation word that is adjacent or within a number of words of a contextual term in the electronic interpretation or the variant thereof.

14. The system of claim 10, further comprising a second context adjuster configured to determine a context of the electronic requisition and determine the rank or score of one or more interpretation words or variants thereof based on the context.

15. The system of claim 10, wherein the first database and the second database are computer-processable collections of medical terms.

16. The system of claim 10, wherein the interpretation ranking or scoring module is further configured to assign an increasingly higher rank or score depending on a seriousness, abnormality, or criticality of a condition, finding, or variation indicated by the respective word or variant thereof.

17. A non-transitory computer-readable medium that stores executable instructions that direct an electronic host device to perform a method for processing data, the method comprising:
receiving an electronic requisition comprising an order for an interpreter to interpret one or more files;
parsing the electronic requisition to identify requisition words in the requisition;
detecting which of the requisition words or variants thereof is/are present in a first database of keywords;
respectively ranking or scoring the requisition word(s) or variant(s) thereof that is/are present in the first database;
generating a cumulative requisition rank or score based on the rank(s) or score(s) of the requisition word(s) or variant(s);
receiving an electronic interpretation generated by the interpreter, the electronic interpretation comprising interpretive data based on the one or more files;
parsing the electronic interpretation to identify interpretation words in the interpretation;
detecting which of the interpretation words or variants thereof is/are present in a second database of keywords;
respectively ranking or scoring the interpretation word(s) or variant(s) thereof that is/are present in the second database;
generating a cumulative interpretation rank or score based on the rank(s) or score(s) of the interpretation word(s) or variant(s); and
determining whether the cumulative interpretation rank or score meets a threshold of the cumulative requisition rank or score.

18. The non-transitory computer-readable medium of claim 17, wherein the method further comprises detecting which of the interpretation words or variants thereof are contextual terms and adjusting the rank or score of any interpretation word that is adjacent or within a number of words of a contextual term in the electronic interpretation or the variant thereof.

19. The non-transitory computer-readable medium of claim 17, wherein the method further comprises determining a context of the electronic requisition and determining the rank or score of one or more interpretation words or variants thereof based on the context.

20. The non-transitory computer-readable medium of claim 17, wherein respectively ranking or scoring the interpretation word(s) or variant(s) thereof comprises assigning an increasingly higher rank or score depending on a seriousness, abnormality, or criticality of a condition, finding, or variation indicated by the respective word or variant thereof.
